(19) United States
(12) Patent Application Publication
(10) Pub. No.: US 2022/0361956 A1
(43) Pub. Date: Nov. 17, 2022

(54) Guiding Medical Instruments During Medical Procedures

(71) Applicant: Westface Medical Inc., Redwood City, CA (US)

(72) Inventors: David B. Smith, Redwood City, CA (US); Rachel Ward Rohlen, Redwood City, CA (US); Rida T. Farouki, Davis, CA (US); Jack R. Swett, Davis, CA (US)

(21) Appl. No.: 17/735,877

(22) Filed: May 3, 2022

Related U.S. Application Data
(60) Provisional application No. 63/183,426, filed on May 3, 2021.

(51) Int. Cl. A61B 34/20

(52) U.S. Cl. CPC A61B 34/20; A61B 2034/2074; A61B 2034/2055

(57) ABSTRACT

Techniques and technologies for guiding medical instruments during medical procedures using real-time imaging technologies are disclosed. A representative apparatus includes a medical instrument, an imaging system, a stage assembly, and a control system. The medical instrument includes an elongated portion configured to be inserted into a body portion and having an optical fiber that includes a tip portion that is extendable beyond a distal end of the elongated portion. The imaging system provides a sampling energy that is emitted from the tip portion. The stage assembly actuates the tip portion to perform scanning of one or more tissues with the sampling energy. The imaging system receives a reflected energy, providing a plurality of one-dimensional arrays of intensity values of the reflected energy. The control system analyzes the plurality of one-dimensional arrays of intensity values to determine a shape and a location of the target tissue, and displays information for guiding the medical instrument into engagement with the target tissue.

### CROSS REFERENCE TO RELATED APPLICATIONS

This patent application claims priority benefits under 35 USC § 119(e) from the following provisional patent application: U.S. Patent Application No. 63/183,426 filed on May 3, 2021, which application is incorporated herein by reference.

### FIELD OF THE DISCLOSURE

The present disclosure relates generally to techniques and technologies for guiding medical instruments during medical procedures using real-time imaging technologies.

### BACKGROUND

Many medical procedures typically involve the insertion of a medical instrument into a patient. Insertion of a needle or a catheter into a vein or other lumen of the patient's body for the administration of medication or the collection of blood are representative examples of such routine medical procedures. Although such procedures are commonplace and indispensable, they nevertheless have an imperfect success rate even among experienced medical practitioners. Accordingly, techniques and technologies that improve the success rate of such ubiquitous medical procedures would provide substantial benefit.

### SUMMARY

The present disclosure teaches techniques and technologies for guiding medical instruments during medical procedures using real-time imaging technologies.

Techniques and technologies in accordance with the present disclosure provide guidance for medical practitioners for guiding medical instruments during medical procedures using real-time imaging. Such guidance information may advantageously improve the performance of medical procedures by enabling such procedures to be performed more accurately, more reliably, and with less repetition in comparison with prior art techniques. Accordingly, techniques and technologies in accordance with the present disclosure may provide substantially improved satisfaction of medical practitioners and patients in comparison with prior art practices and procedures.

More specifically, in at least some implementations, an apparatus for guiding medical instruments during medical procedures comprises a medical instrument, an imaging system, a stage assembly, and a control system. The medical instrument may include an elongated portion configured to be inserted into a body portion of a patient and having an optical fiber disposed within the elongated portion. The optical fiber has a tip portion that is extendable beyond a distal end of the elongated portion. The imaging system is configured to provide a sampling energy into the optical fiber, the sampling energy being emitted from the tip portion along a sampling vector at a deflection angle with the longitudinal axis as the elongated portion is inserted into the body portion. The stage assembly may actuate the optical fiber and the tip portion to perform a scanning of one or more tissues with the sampling energy emitted from the tip portion. More specifically, the stage assembly is configured to rotate the tip portion about a scanning axis that is parallel with the longitudinal axis and to reciprocate the tip portion along the scanning axis that is parallel with the longitudinal axis with the tip portion proximate to and extended beyond the distal end of the elongated portion. The sampling energy is emitted from the tip portion in a scanning pattern. The imaging system receives a reflected energy that is reflected from the one or more tissues back through the optical fiber, providing a plurality of one-dimensional arrays of intensity values of the reflected energy at a plurality of distances along the sampling vector. The control system analyzes the plurality of one-dimensional arrays of intensity values of the reflected energy to determine a shape of a target tissue and a location of the target tissue, and displays information including one or more relative positions of the target tissue relative to the elongated portion of the medical instrument for guiding the medical instrument into engagement with the target tissue as the elongated portion is inserted into the body portion for performance of the medical procedure.

Similarly, in at least some implementations, a method for performing a medical procedure in accordance with the present disclosure includes: engaging a medical instrument with a body portion of a patient, the medical instrument including an elongated portion configured to be inserted into the body portion and having an optical fiber at least partially disposed within the elongated portion, the optical fiber having a tip portion that is extendable beyond a distal end of the elongated portion; actuating an imaging system that provides a sampling energy into the optical fiber, the sampling energy being emitted from the tip portion along a sampling vector at a deflection angle with the longitudinal axis into one or more tissues of the body portion as the elongated portion is inserted into the body portion, the one or more tissues including a target tissue; and actuating a stage assembly to move the tip portion to perform a scanning of the one or more tissues with the sampling energy emitted from the tip portion, the stage assembly rotating at least the tip portion about a scanning axis that is parallel with the longitudinal axis and reciprocating at least the tip portion along the scanning axis that is parallel with the longitudinal axis with the tip portion proximate to and extended beyond the distal end of the elongated portion, the sampling energy being emitted from the tip portion in a scanning pattern. The method further includes receiving a reflected energy that is reflected from the one or more tissues back through the optical fiber to the imaging system, the reflected energy providing a plurality of one-dimensional arrays of intensity values of the reflected energy at a plurality of distances along the sampling vector; analyzing the plurality of one-dimensional arrays of intensity values of the reflected energy to determine a shape of the target tissue and a location of the target tissue relative to the elongated portion of the medical instrument; and displaying information including one or more relative positions of the target tissue relative to the elongated portion of the medical instrument for guiding the medical instrument into engagement with the target tissue as the elongated portion is inserted into the body portion for performance of the medical procedure.

This summary is intended to provide an introduction of a few exemplary aspects of implementations in accordance with the present disclosure. It is not intended to provide an exhaustive explanation of all possible implementations, and should thus be construed as merely introductory, rather than limiting, of the following disclosure.

### BRIEF DESCRIPTION OF THE DRAWINGS

FIG. 1 shows a schematic view of a system for performing a medical procedure using real-time imaging in accordance with an embodiment of the present disclosure.

FIG. 2 shows a schematic view of an imaging system of the system of FIG. 1 in accordance with an embodiment of the present disclosure.

FIG. 3 shows a schematic view of a stage assembly of the system of FIG. 1 in accordance with an embodiment of the present disclosure.

FIG. 4 shows a schematic view of a handheld component of the system of FIG. 1 in accordance with an embodiment of the present disclosure.

FIG. 5 shows a coordinate system associated with the handheld component of the system of FIG. 1 in accordance with an embodiment of the present disclosure.

FIG. 6 shows a process for performing a medical procedure using real-time imaging in accordance with an embodiment of the present disclosure.

FIG. 7 shows a needle of the handheld component positioned in a first position proximate to the body portion of the patient in accordance with an embodiment of the present disclosure.

FIG. 8 shows a representative image acquired by the imaging system during a scanning operation in accordance with an embodiment of the present disclosure.

FIG. 9 shows a first view displayed by a display device of the control system during a medical procedure in accordance with an embodiment of the present disclosure.

FIG. 10 shows a second view displayed by the display device of the control system during the medical procedure in accordance with an embodiment of the present disclosure.

FIG. 11 shows the needle of the handheld component positioned in a second position entering the vein of the body portion of the patient in accordance with an embodiment of the present disclosure.

FIG. 12 shows a third view displayed by the display device of the control system during the medical procedure in accordance with an embodiment of the present disclosure.

FIG. 13 shows the needle of the handheld component positioned in a third position fully entered within the vein of the body portion of the patient in accordance with an embodiment of the present disclosure.

FIG. 14 shows a fourth view displayed by the display device of the control system during the medical procedure in accordance with an embodiment of the present disclosure.

FIG. 15 shows a fifth view displayed by the display device of the control system during the medical procedure in accordance with an embodiment of the present disclosure.

FIG. 16 shows a first computed cylinder based on the computational results of a first example simulation in accordance with the present disclosure.

FIG. 17 shows a second computed cylinder based on the computational results of a second example simulation in accordance with the present disclosure.

FIG. 18 shows a third computed cylinder based on the computational results of a third example simulation in accordance with the present disclosure.

FIG. 19 is a schematic view of an exemplary computing device configured to operate in accordance with the present disclosure.

### DETAILED DESCRIPTION

Techniques and technologies for guiding medical instruments during medical procedures using real-time imaging technologies will now be disclosed. In the following description, many specific details of certain implementations are described and shown in the accompanying figures. One skilled in the art will understand that the present disclosure may have other possible implementations, and that such other implementations may be practiced with or without some of the particular details set forth in the following description.

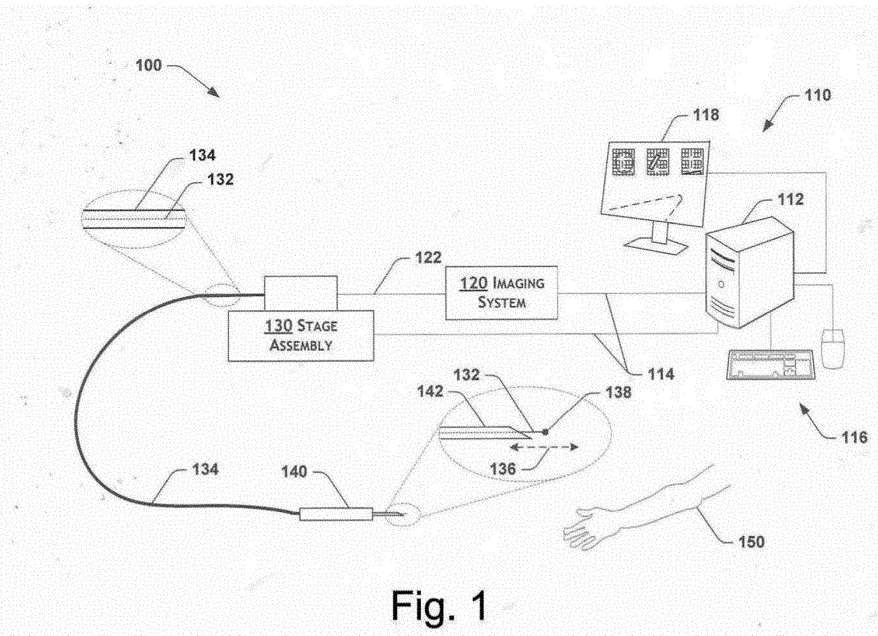

FIG. 1 shows a schematic view of a system 100 for performing a medical procedure using real-time imaging in accordance with an embodiment of the present disclosure. In this embodiment, the system 100 includes a control system 110 operatively coupled to an imaging system 120 and a stage assembly 130. The control system 110 includes a computing component 112 that transmits control signals via conductive leads 114 to control operation of the imaging system 120 and the stage assembly 130. More specifically, the control system 110 may include logic in the form of software, firmware, hardware, or combinations thereof, that collects imaging data from the imaging system 120 and position data from the stage assembly 130, and performs geometric reconstructions described below. In the embodiment shown in FIG. 1, the control system 110 includes one or more user input devices 116 (e.g. keyboard, mouse, etc.) to enable user operation of the system 100, and a display device 118 for displaying one or more real-time images 160 for guiding medical instruments during medical procedures as described more fully below.

The system 100 further includes a first optical fiber 122 that extends from the imaging system 120 to the stage assembly 130, and a second optical fiber 132 that extends within a sheath 134 to a handheld component 140. The sheath 134 is preferably a flexible sheath that surrounds the second optical fiber 132 and enables a user (e.g. a nurse, medical practitioner, etc.) to move within the environment for positioning the handheld component 140 at one or more desired locations proximate to a body portion 150 of a patient (e.g. an arm). In the embodiment shown in FIG. 1, the handheld component 140 includes a needle 142 that is configured for insertion into the body portion 150 of the patient, such as for intravenous administration of a medication. The second optical fiber 132 is loosely captured within the sheath 134 and is free to translate along its longitudinal axis within the sheath 134 (as indicated by double arrow 136) such that a tip 138 of the second optical fiber 132 may be extended through the handheld component 140 and beyond an end of the needle 142, or may be retracted toward the handheld component 140 into the needle 142.

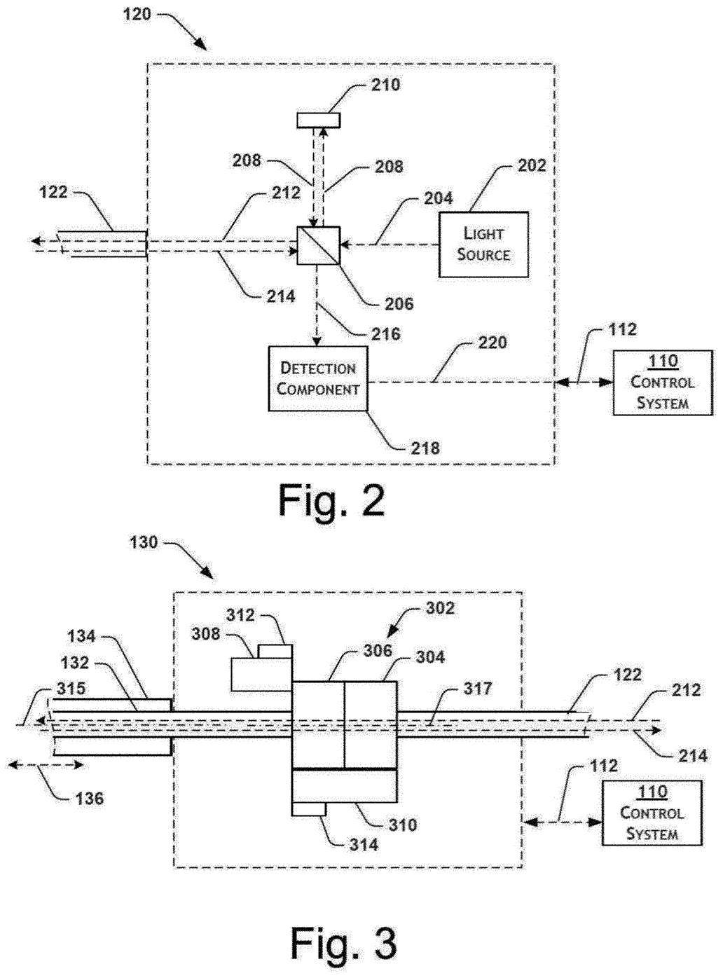

The imaging system 120 may be any of a wide variety of suitable imaging systems. For example, FIG. 2 shows a schematic view of the imaging system 120 in accordance with an embodiment of the present disclosure. In this embodiment, the imaging system 120 is based on optical coherence tomography. Optical coherence tomography (OCT) is a well-documented imaging modality that may use low-coherence interferometry to obtain three-dimensional, relatively high-resolution images of biological tissue at depths of a few millimeters. It will be appreciated that OCT systems may be configured in a variety of suitable configurations, and that the imaging system 120 shown in FIG. 2 is simply one possible embodiment suitable for use with the system 100.

In the embodiment shown in FIG. 2, the imaging system 120 includes a light source 202 that emits light 204 toward a deflector component 206. Preferably, the light source 202 is a broad-spectrum light source in the near infrared or infrared band to enable optimal depth of penetration into human tissue. The deflector component 206 splits the incoming light 204 into a reference light 208 that is reflected toward a reference mirror 210, and a sampling light 212 that is emitted out of the imaging system 120 into the first optical fiber 122. The deflector component 206 may include one or more interferometers or optical interference systems that operate as described herein, such as a beam splitter, an isolator, a coupler, a circulator, a partially severed mirror with one or more holes therein, or any other suitable components.

As shown in FIG. 2, the reference light 208 is reflected by the reference mirror 210 back to the deflector component 206. The sampling light 212 exits the imaging system 120 and transits via the stage assembly 130 and the second optical fiber 132 (shown in FIG. 1) toward a sample that is to be imaged, such a tissue of the body portion 150 of the patient. A portion of the sampling light 212 is reflected from the sample and returns to the imaging system 120 (via the second optical fiber 132 and the stage assembly 130) as a reflected light 214. Because the reflected light 214 and the reference light 208 have traveled different optical path lengths, the reflected light 214 and the reference light 208 combine (or re-combine) at the deflecting component 206 to generate a combined light 216 that includes an interference (or fringe) pattern that is received by a detection component 218. The detection component 218 may include, for example, one or more photodiodes, photodetectors, multi-array cameras, or other suitable components. The detection component 218 senses the combined light 216 and transmits an imaging signal 220 that is output from the imaging system 120 to the control system 110 for further processing. Using known, well-established processing techniques (e.g. Fourier analysis, etc.), the control system 110 may process the imaging signal 220 output from the imaging system 120 into one or more images for display on the display device 118, as described more fully below.

It should be appreciated that the imaging system 120 shown in FIG. 2 is just one possible embodiment of an OCT-based imaging system that is suitable for use in the system 100, and that numerous alternate embodiments may be conceived and employed. For example, suitable alternate embodiments of OCT-based imaging systems are described in U.S. Pat. No. 10,323,926 issued to Elmaanaoui et al., U.S. Pat. No. 9,593,935 issued to Osawa et al., U.S. Pat. No. 8,811,702 issued to Kurosaka et al., In Vivo Endoscopic Optical Biopsy with Optical Coherence Tomography by Guillermo et al., published in the journal Science at vol. 276:2037-2039, and in High Resolution In Vivo Intra-Arterial Imaging With Optical Coherence Tomography by Fujimoto et al., published in the journal Heart 1999 at vol. 82:128-133, which publications are incorporated herein by reference.

Furthermore, it should be appreciated that alternate imaging systems other than OCT-based imaging systems may also be suitable for use in the system 100. Imaging systems that employ other imaging modalities that collect a set of three-dimensional surface data in order to identify a cylindrical object may also be employed in accordance with the teachings of the present disclosure. More specifically, in at least some implementations, such alternate imaging systems may operate in accordance with the present disclosure by being capable of providing a one dimensional array of signal intensities along a vector in three dimensional space, originating from the tip 138 of the second optical fiber 132 and extending a suitable distance (e.g. a few millimeters) forward therefrom, as will be described more fully below.

Similarly, the stage assembly 130 may be any one of a wide variety of suitable assemblies. For example, FIG. 3 shows a schematic view of the stage assembly 130 of the system 100 of FIG. 1 in accordance with an embodiment of the present disclosure. In this embodiment, the stage assembly 130 includes a fiber optic rotary joint (FORJ) 302 having a first portion 304 coupled to the first optical fiber 122 and a second portion 306 coupled to the second optical fiber 132. The sampling light 212 emitted by the imaging system 120 enters the stage assembly 130 via the first optical fiber 122, traverses through the fiber optic rotary joint 302, and exits from the stage assembly 130 through the second optical fiber 132. Similarly, the reflected light 214 that is reflected from the sample (e.g. a tissue of the body portion 150 of the patient) enters the stage assembly 130 via the second optical fiber 132, traverses through the fiber optic rotary joint 302, and exits from the stage assembly 130 through the first optical fiber 122 en route to the imaging system 120.

As further shown in FIG. 3, a rotation stage motor 308 is operatively coupled to the second portion 306 of the fiber optic rotary joint 302 to cause rotation of the second portion 306 (and the second optical fiber 132) relative to the first portion 304. In addition, a translation stage motor 310 is operatively coupled to the fiber optic rotary joint 302 to cause translation of the fiber optic rotary joint 302 (and the second optical fiber 132) in forward and rearward directions along a longitudinal axis 315 of the second optical fiber 132 (as indicated by double arrow 136). At a first end of the second optical fiber 132 proximate to the stage assembly 130, the longitudinal axis 315 of the second optical fiber 132 is at least approximately collinear (or coincident) with a longitudinal axis 317 of the FORJ 302. A rotation encoder 312 monitors rotational position and rate of the second portion 306 (and the second optical fiber 132), while a translation encoder 314 monitors translational position and rate of the second portion 306 (and the second optical fiber 132). More specifically, in at least some implementations, the translation encoder may monitor the translation of the entire FORJ 302 and the second optical fiber 132, as the second portion 306 has a rotational degree of freedom, but no translational degree of freedom, with respect to the first portion 304. The control system 110 provides control signals to the stage assembly 130 to control the rotation stage and translation stage motors 308, 310, and the rotation and translation encoders 312, 314 provide feedback to the control system 110 to achieve precise control of the rotational and translational movements of the tip 138 of the second optical fiber 132 (FIG. 1). In at least some implementations, the sheath 134 is not rotated or translated by the stage assembly 130, but rather, the second optical fiber 132 is free to rotate and translate within the sheath 134

More specifically, in at least some embodiments, the rotation stage motor 308 rotates the second portion 306 and the second optical fiber 132 at a fixed controllable rate, e.g. 10 Hz, around the axis of the second optical fiber 132. The rotation encoder 312 tracks the angular position of the rotation stage motor 308 and sends a timestamped “index pulse” to the control system 110 at each full revolution. In addition, in at least some embodiments, the translation stage motor 310 moves the fiber optic rotary joint 302 (and the second optical fiber 132) forward and backwards, parallel to the longitudinal axis of the second optical fiber 132, reciprocating along a triangular waveform. The control system 110 may control the stage assembly 130 to controllably adjust the rate and range of translation of the tip 138 of the second optical fiber 132 to any suitable values. For example, in at least some implementations, the translation rate may have a value within a range of approximately 0.25 Hz to approximately 2.0 Hz, and the translation range may have a value within a range of approximately 2 millimeters to approximately 8 millimeters. In a particular embodiment, the stage assembly 130 translates the tip 138 of the second optical fiber 132 along a translation range of 6 mm at a rate of approximately 0.5 Hz. The translation encoder 310 sends continuous time stamped position information to the control system 110. As noted above, the second optical fiber 132 passes through and moves within the sheath 134, but note that the sheath 134 is not translated or rotated by the stage assembly 130, and that, in at least some implementations, a proximal end of the sheath 134 remains fixed with respect to the stage assembly 130.

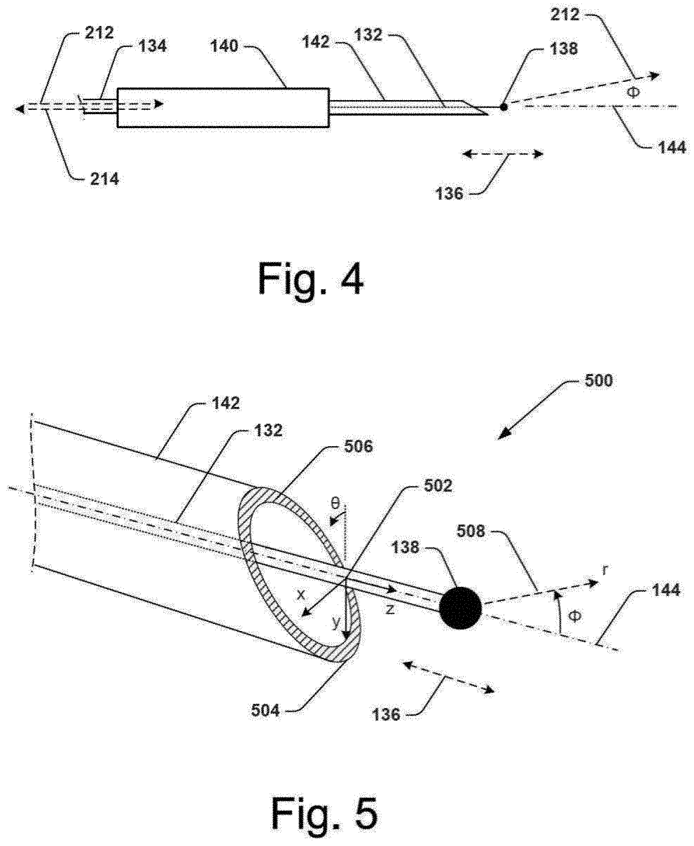

FIG. 4 shows a schematic view of the handheld component 140 of the system 100 of FIG. 1 in accordance with an embodiment of the present disclosure. The handheld component 140 is connected to the sheath 134 but is not directly connected to the second optical fiber 132 so that the handheld component 140 and the needle 142 are not affected by the rotational and translational motion of the second optical fiber 132. As noted above, the second optical fiber 132 is configured to translate along the longitudinal axis 315 of the second optical fiber 132 (as indicated by double arrow 136) such that the tip 138 of the second optical fiber 132 may be extended through the handheld component 140 and beyond the end of the needle 142. At locations proximate to the tip 138, in at least some implementations, the longitudinal axis 315 of the second optical fiber 132 is collinear (or coincides) with a longitudinal axis 144 of the needle 142. Therefore, the stage assembly 130 rotates and translates the tip 138 of the second optical fiber 132 along the longitudinal axis 144 relative to the needle 142. As further shown in FIG. 4, the tip 138 is configured so that the sampling light 212 traversing the second optical fiber 132 is emitted by the tip 138 at a deflection angle phi (or 4) with respect to the longitudinal axis 144 of the needle 142. For example, in at least some embodiments, the tip 138 contains a lens that deflects the sampling light 212 along the deflection angle phi.

Although the handheld component 140 is described herein as having the needle 142 for intravenous insertion into the body portion 150 of the patient, it should be appreciated that the handheld component 140 may have alternate configurations. For example, in alternate embodiments, the handheld component 140 may be a catheter that is configured for insertion into any suitable lumen or body portion.

The operations performed by the system 100 for guiding medical instruments during medical procedures using real-time imaging technologies may use a variety of suitable reference systems. For example, FIG. 5 shows a coordinate system 500 associated with the handheld component 140 of the system 100 of FIG. 1 in accordance with an embodiment of the present disclosure. In this embodiment the coordinate system 500 is a three-dimensional (3D) cartesian coordinate system defined by the distal end of the needle 142. Although the needle may move while the target remains stationary in practice, in at least some implementations, the coordinate system 500 may be defined such that the needle 142 remains stationary with respect to the coordinate system 500 and all else moves around it. The origin 502 lies on the longitudinal axis 144 (of the needle 142 and the approximately collinear second optical fiber 132), with the z axis collinear to the longitudinal axis 144 and pointed distally, such that a distal-most point 504 of a needle bevel 506 lies in the xy plane (z=0). The y axis is defined pointing directly towards the distal-most point (or bevel tip) 504, while the x axis is normal to y and z according to the right hand rule. For further simplification, the positive x direction is referred to as “right”, with positive y and positive z referred to as “down” and “forward” respectively.

In at least some implementations, the position of the second optical fiber 132 (and the tip 138) may be defined using a mix of cartesian and radial coordinate conventions. First, the longitudinal axis 144 of the second optical fiber 132 is collinear to the axis of the needle 142 (i.e. z axis) as previously defined. Activity by the translation stage motor 310 of the stage assembly 130 will translate the tip 138 forward and back along the z axis, with the translation encoder 314 tracking the z value of the tip 138. As noted above, the tip 138 will deflect the sampling light 212 along a light vector 508 transmitted from the tip 138 at the deflection angle phi with respect to the z axis (see FIG. 4). In at least some embodiments, the deflection angle phi may be 60 degrees, although other suitable angles may be used. In at least some implementations, for example, the deflection angle phi may have a fixed value within a range of approximately 30 degrees to approximately 90 degrees.

Furthermore, the rotation motor 308 rotates a light vector of the sampling light 212 around the z axis by a rotation angle theta (or Θ). By convention, the rotation encoder 312 defines the rotation angle theta=0 when the exit vector (or light vector 508) lies in the yz plane and the exit vector has a negative y component. In other words, when rotation angle theta=0, the light vector 508 points “up” as shown in FIG. 5. In at least some implementations, the rotation encoder 312 sends the aforementioned index pulse every time theta crosses 0 degrees, indicating one revolution of the second optical fiber 132.

From the aforementioned values, the light vector 508 of the sampling light 212 emitted by the tip 138 of the second optical fiber 132 may be defined. The light vector 508 originates from a cartesian position (0,0,z0), with the value z0 controlled by the translation motor 310 and recorded by the translation encoder 314. The light vector 508 always forms the deflection angle phi with the z axis that remains constant, and a projection of the light vector 508 in the xy plane makes the rotation angle theta with respect to the −y axis (see FIG. 5). The imaging system 120 will measure signal intensity at several distances “r” along the length of the light vector 508, with distances “r” ranging from 0 to a few millimeters in the case of OCT. Therefore, any measured position in space defined by the ordered triplet (r, theta,z0) can be resolved to a cartesian value (X,Y,Z).

Although for the sake of clarity, the foregoing discussion has described the tip 138 of the second optical fiber 132 as being located on the longitudinal axis 144 of the needle 142, it will be appreciated that this is merely one possible implementation, and that in alternate implementations, the second optical fiber 132 and the tip 138 may be radially offset from the longitudinal axis 144. For example, in at least some implementations, the second optical fiber 132 may be positioned along a sidewall of the needle 142 such that the tip 138 is offset from the longitudinal axis 144 by a predetermined radial distance. During data processing (analysis and imaging) operations described herein, such an offset of the tip 138 from the longitudinal axis 144 may be readily taken into account in the data reduction algorithms performed by the control system 110. Thus, whenever the tip 138 of the second optical fiber 132 is described and shown in the accompanying figures as being located on the longitudinal axis 144 of the needle 142, it will be appreciated that this is one possible implementation, and that in alternate implementations the tip 138 may be offset from the longitudinal axis 144 by a predetermined radial distance. In such alternate implementations, a more generalized description applies, wherein the tip 138 is rotated by the stage assembly 130 about a scanning axis that is parallel with the longitudinal axis 144 of the needle 142, and is reciprocated by the stage assembly 130 along the scanning axis that is parallel with the longitudinal axis 144 of the needle 142. In at least some implementations, as previously noted, the scanning axis is collinear with the longitudinal axis 144 of the needle 142 (or other elongated medical instrument).

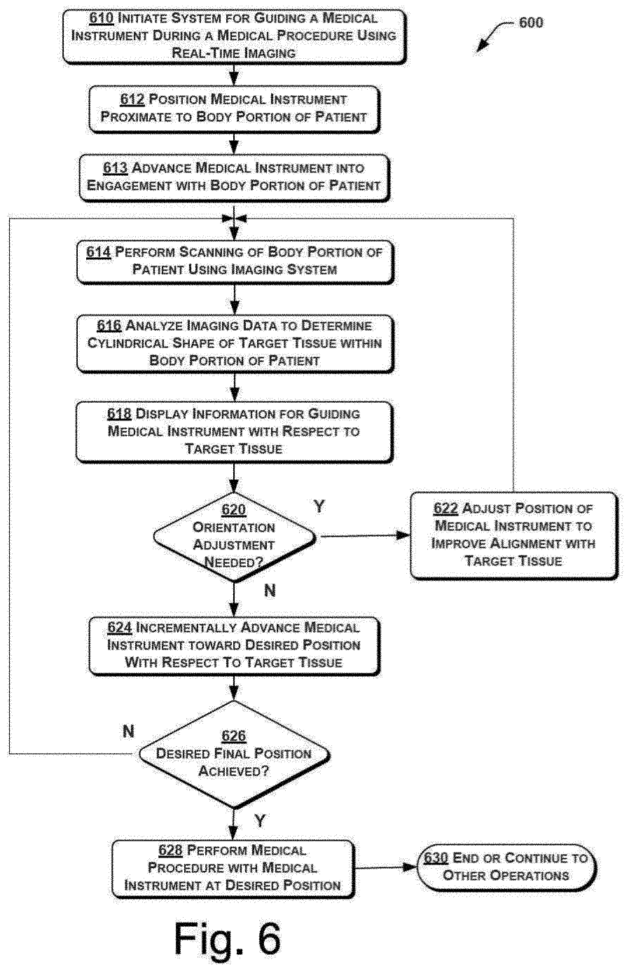

FIG. 6 shows a process 600 for performing a medical procedure using real-time imaging in accordance with an embodiment of the present disclosure. In this embodiment, the process 600 includes initiating a system (e.g. system 100 of FIGS. 1-5) for guiding a medical instrument during a medical procedure using real-time imaging at 610. More specifically, in at least some implementations, the initiating (at 610) includes activating the imaging system 120 and the stage assembly 130 of the system 100, such as by causing the control system 110 to transmit control signals to these components so that the tip 138 is rotated about the z axis, and the sampling light 212 provided by the imaging system 120 is emitted from the tip 138, as shown in FIG. 5. In at least some implementations, the initiating (at 610) does not include causing the stage assembly 130 to translate the tip 138 in a reciprocating manner, and the tip 138 may remain withdrawn into the needle 142 during the initiating (at 610).

The process 600 further includes positioning a medical instrument proximate to a body portion of a patient at 612. For example, in at least some implementations, the positioning of the medical instruction (at 612) may include a healthcare provider positioning the handheld component 140 of the system 100 so that the needle 142 is proximate to an arm or other body portion 150 of the patient.

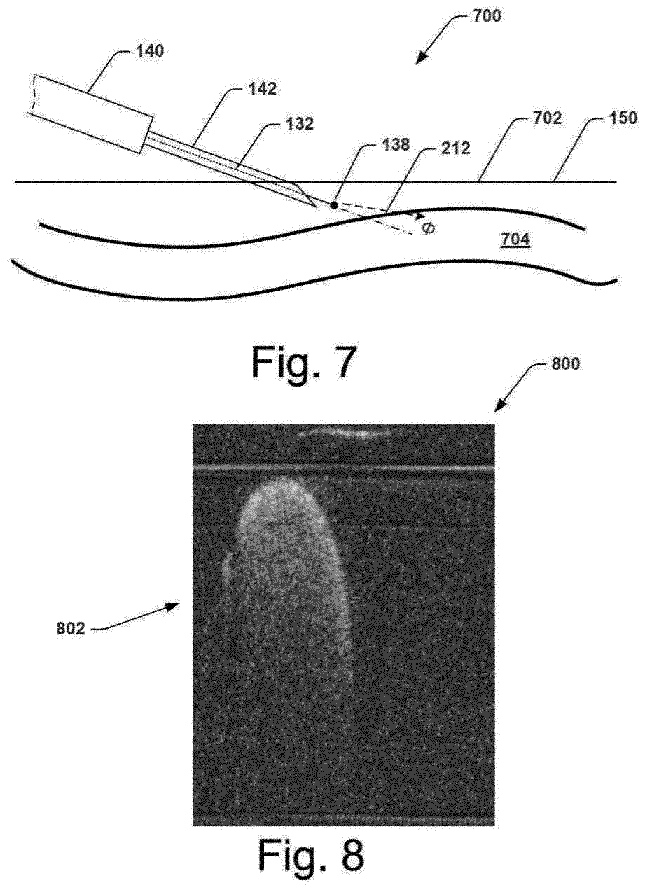

Next, the process 600 includes advancing the medical instrument into engagement with a body portion of the patient at 613. In at least some implementations, the advancing (at 613) includes advancing the handheld component 140 until the needle 142 starts to penetrate a skin surface of the body portion 150 of the patient. More specifically, FIG. 7 shows the needle 142 of the handheld component 140 positioned in a first position 700 with respect to the body portion 150 of the patient. In the first position 700, the needle 142 has penetrated the skin 702 of the body portion 150.

As further shown in FIG. 6, the process 600 further includes performing scanning of the body portion 150 of the patient at 614. For example, in at least some implementations, once the medical instrument has been advanced to a suitable position for scanning (e.g. the needle 142 has penetrated the skin 702 as shown in FIG. 7), the control system 110 may activate the translation motor 310 of the stage assembly 130 so that the tip 138 of the second optical fiber 132 is advanced beyond the bevel tip 504 of the needle 142, and reciprocating translation is initiated, causing the tip 138 to both rotate and reciprocate as indicated by double arrow 136 of FIG. 5. During the performing of the scanning (at 614), the sampling light 212 emitted by the tip 138 of the second optical fiber 132 may impinge upon and penetrate the tissues of the body portion 150 such that a target vein 704 located below the skin 702 (FIG. 7) is at least partially illuminated by the sampling light 212. The reflected light 214 may then be reflected from the target vein 704 and other tissues of the body portion 150 and returned along the second optical fiber 132 (and first optical fiber 130) to the imaging system 120. Accordingly, in at least some implementations, the scanning (at 614) includes collecting and recording intensity data associated with the reflected light 214 that is reflected from the tissue(s) of the body portion 150 and returned to the imaging system 120 (and to the control system 110), as described more fully below.

It will be appreciated that in alternate implementations, operations of the process 600 may be altered, combined, or performed in a different order. For example, the initiating of the rotation of the tip 138 by the stage assembly 130, and the initiating of the imaging system 120, may occur during the performing of the scanning (at 614) rather than at the initiating of the system (at 610). Thus, the operations described herein may be combined or performed in a different order without departing from the scope of the present disclosure.

In addition, it should be appreciated that a variety of suitable scanning procedures (at 614) may be employed. For example, in at least some implementations, the rotation motor 308 rotates the second optical fiber 132, and consequently the light vector 508 emitted from the tip 138, along the rotation angle theta at a constant rate, while the translation motor 310 reciprocates the linear position of the tip 138 along the z axis (i.e. value z0) in a triangular waveform. This causes the light vector 508 (FIG. 5) of sampling light 212 to sweep a reciprocating helical pattern into the tissues in the region near the tip 138 and correspondingly near a tip of the medical instrument, such as the bevel 506 of the needle 142 shown in FIG. 5. The translation motion may be controllably adjusted (e.g. by the control system 110) so that z0 tracks from z=0 to some suitable positive value. Similarly, the rotational velocity of the rotation motor 308 may be controllably adjusted such that the tip 138 performs several revolutions about the rotation angle theta for each leg of the translational motion of the tip 138.

In addition, in at least some implementations, the imaging system 120 (and control system 110) continuously captures a one-dimensional array (or “aLine”) of signal intensity at various distances r along the light vector 508. Each aLine captured during rotation of the tip 138 at successive rotational angles theta corresponds to the same domain of r values, but will have different values for theta and z0 caused by the rotation and translation motors 308, 310. The control system 110 may collect aLines continuously, but may separate them into batches as the rotation encoder 312 transmits index pulses, with each index pulse corresponding to one revolution of the rotation motor 308 (or tip 138). These batches are referred to as bScans. Each bScan may be rendered into an image, with each column in the image representing a single aLine. Each row in the image has the same r value, which is determined by OCT physics and does not change between aLines. Based on the timestamps of the incoming aLines, interpolated with the timestamps of the linear encoder data, each aLine is assigned a z0 value. Based on the timestamps of the incoming aLines, interpolated with the timestamps of the rotation encoder 312 index pulses, and assuming a constant rotational velocity of the rotation motor 308, each column is assigned a theta value. Therefore, for any pixel in any bScan, values of (r, theta,z0) and corresponding voxels with cartesian coordinates (X,Y,Z) may be calculated.

For example, FIG. 8 shows a representative image 800 acquired by the imaging system during a scanning operation (at 614) in accordance with an embodiment of the present disclosure. More specifically, the representative image 800 shows a representative bScan of intensity data in a rasterized configuration. In this representative image 800, the target vein (or blood vessel) 704 is represented by the relatively brighter (or “shark fin”) shape 802. The position of the tip 138 is approximately along a top row of the image 800. Scanning from top to bottom, an outer wall of the target vein (or blood vessel) 704 is approximately along a top edge of the relatively brighter shape 802, while an interior lumen of the target vein (or blood vessel) 704 is along an underside of the shape 802 as the intensity fades to black.

Referring again to FIG. 6, the process 600 further includes analyzing imaging data (collected during scanning operations at 614) to determine a cylindrical shape of the target tissue within the body portion of the patient at 616. For example, in at least some implementations, the intensity data gathered in the form of bScans during the scanning operations (at 614) are analyzed using the control system 110 to approximate the target vein 704 as a cylindrical shape in the region near the tip 138 in real-time during the performance of the medical procedure.

More specifically, using image processing techniques (discussed more fully below), each bScan is analyzed to identify the pixels that contain the “outer” surface of the target vein 704 (FIG. 7) (or other blood vessel or target structure) within the body portion 150 of the patient. These pixels may be assembled in list format and the (X,Y,Z) voxels are identified. As the system 100 collects multiple bScans, the system 100 may store a rolling list of voxels (e.g. in memory of the control system 110) from a desired value “n” of bScans (e.g. n=5). In at least some implementations, each time the list may be updated by adding a new bScan and purging the oldest stored bScan, the processing techniques performed by the control system 110 may determine a cylinder geometry of the target vein 704 (or other target tissue) by inputting the ordered triplets, (e.g. starting at Equation 14, described more fully below). In addition, processing techniques performed by the control system 110 will calculate the parameters of the cylinder of the target vein 704 (or target tissue), including an axis and radius. In at least some implementations, the cylindrical shape of the target vein 704 may be defined (at 616) using five parameters as follows. A central axis of the cylinder is defined as passing through the cartesian points (X0,Y0,0) and (X1,Y1,1), and the cylinder has a radius R.

Referring again to FIG. 6, the process 600 further includes displaying information for guiding the medical instrument with respect to the target tissue at 618. For example, in at least some implementations, the displaying (at 618) includes transmitting the calculated values of the five parameters (X0,Y0,X1,Y1,R) to a visualization program of the control system 110 that renders the cylindrical shape of the target vein 704 in a graphic display relative to the needle 142 of the handheld component 140, and then displaying a real-time image 160 on the display device 118 of the control system 110 that assists the medical practitioner in guiding the needle 142 during performance of the medical procedure.

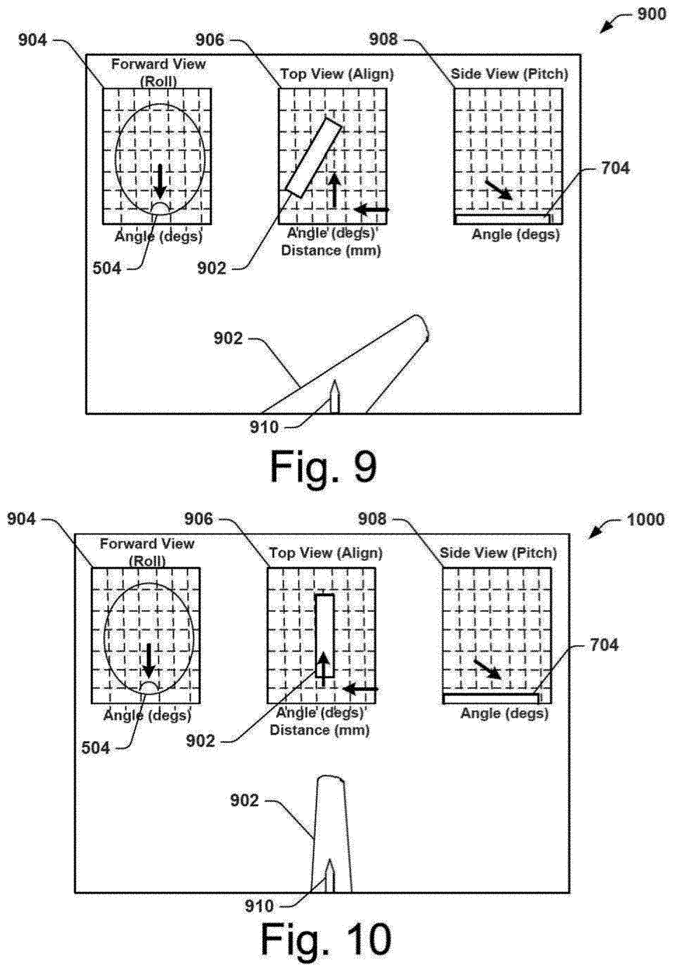

FIG. 9 shows a first view 900 displayed by the display device 118 of the control system 110 during the medical procedure in accordance with an embodiment of the present disclosure. In at least some implementations, the first view 900 includes a displayed blood vessel 902, which is a representation of the computed cylindrical shape of the vein 704 resulting from the analysis (at 616) as the handheld component 140 is positioned at the first position 700 proximate to the vein 704 (FIG. 7). The first view 900 also includes a displayed needle 910, which is a representation of the needle 142 (or other medical instrument).

As further shown in FIG. 9, the first view 900 may include a forward view 904 which shows the alignment of the bevel 506 and the bevel tip 504, providing “roll” guidance to the user as to which way to roll (or rotate) the handheld component 140 (e.g. in degrees) to place the bevel tip 504 at the desired position (e.g. bevel tip 504 closest to the skin 702 of the body portion 150, or bevel 506 up, etc.). In at least some implementations, the “roll” position of the medical instrument (e.g. needle 142) may not be critical to the medical procedure such that the medical practitioner may keep the roll orientation relatively constant over the course of the medical procedure.

With continued reference to FIG. 9, a top view 906 may also be included which provides alignment guidance to the user, showing whether the needle 142 is aligned (in the xz plane) with the longitudinal axis of the vein 704. The top view 906 may also provide distance to the vein 704 (e.g. in millimeters) and angular mis-alignment (e.g. in degrees). Similarly, a side view 908 may also be included which provides pitch alignment guidance to the user, showing the angle at which the needle 142 is oriented (in the yz plane) with respect to the vein 704 (e.g. in degrees). Thus, the guidance information provided by the first view 900 displayed to the medical practitioner (at 618) may advantageously inform the medical procedure of whether the needle 142 (or other medical instrument) is properly aligned with the target tissue for performance of the medical procedure.

With continued reference to FIG. 6, the process 600 further includes determining whether an orientation adjustment of the medical instrument is needed at 620. For example, the medical practitioner may assess the information shown in the first view 900 (FIG. 9) and decide that an adjustment is needed in one or more of the roll, align, or pitch orientations of the needle 142.

If it is determined that an orientation adjustment is needed (at 620), then the process 600 proceeds to adjusting a position of the medical instrument to improve alignment with the target tissue at 622. For example, based on the first view 900 shown in FIG. 9, the medical practitioner may decide to adjust the alignment of the needle 142 (based on the top view 906), but may choose not to adjust the roll or pitch orientations of the needle 142. Alternately, the adjusting (at 622) could include translation to fix roll, e.g., if the vessel is off to one side (on view 904) but “crosses” in front of the needle according to view 906, the practitioner may adjust (at 622) by advancing the needle 142 until it is above the vessel 704, then yaw to align, then pitch down. The process 600 then returns to scanning of the body portion (at 614), and the operations 614 through 620 are then repeated.

FIG. 10 shows a second view 100 displayed by the display device 118 of the control system 110 during the medical procedure in accordance with an embodiment of the present disclosure. Since the scanning (at 614), analyzing (at 616), and displaying (at 618) operations may continue to be continuously performed during the orientation adjustment operation (at 622), the second view 100 of the display device 118 may advantageously show the adjustments of the needle 142 as they are being made by the medical practitioner. Thus, in the second view 100, the top view 906 shows that the needle 142 has been brought into proper alignment (in the the yz plane) with respect to the vein 704

Once it is determined that no orientation adjustment is needed (at 620, then the process 600 proceeds to incrementally advancing the medical instrument toward a desired position with respect to the target tissue at 624. More specifically, the medical practitioner may slowly advance the needle 142 a relatively short distance (e.g. a millimeter) toward the vein 704.

As further shown in FIG. 6, the process 600 may then determine whether a desired final position of the medical instrument has been achieved at 626. For example, in at least some implementations, the medical practitioner decides whether the needle 142 has been sufficiently inserted into the vein 704 to perform the medical procedure (e.g. intravenous administration of medication). If it is determined that the desired final position of the medical instrument has not been achieved (at 626), then the process 600 then returns to scanning of the body portion (at 614), and the operations 614 through 620 are then repeated. In this way, the process 600 continuously performs scanning operations (at 614), analyzing imaging data (at 616), displaying guidance information (at 618), determining whether orientation adjustments are needed (at 620), performing orientation adjustments (at 622), and incrementally advancing the medical instrument (at 626), until the desired final position of the medical instrument is achieved (at 626).

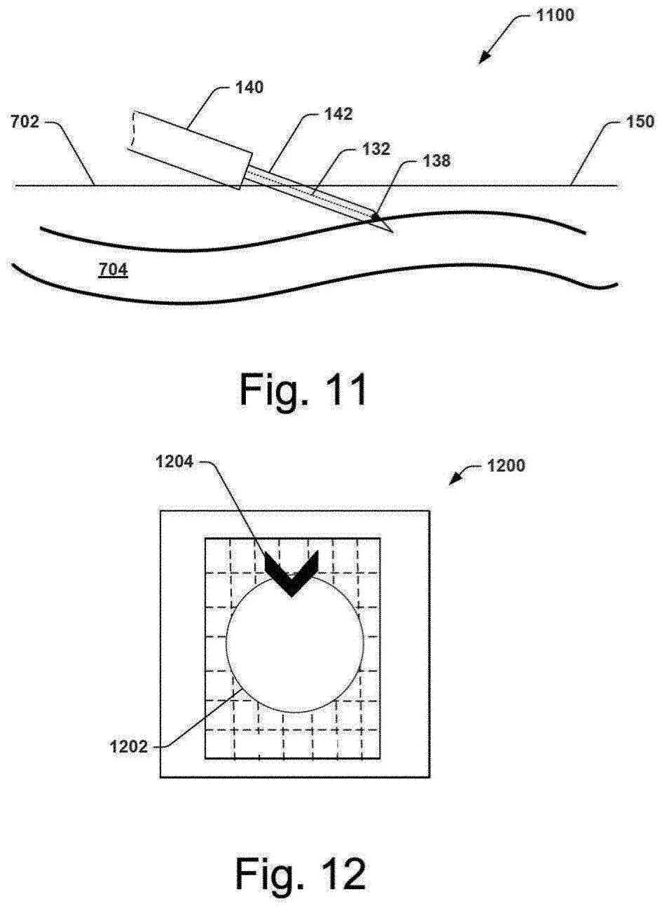

As the medical instrument is being successively advanced (at 624) toward the target tissue, there may be a point at which contact with the target tissue is made. For example, FIG. 11 shows the needle 142 of the handheld component 140 positioned in a second position 1100 such that the bevel 506 (and bevel tip 504) have started entering the vein 704 of the body portion 150 of the patient in accordance with an embodiment of the present disclosure. In at least some implementations, when the needle 142 makes contact with the vein 704, the control system 110 may retract the tip 138 of the second optical fiber 132 into the needle 142 so that the tip 138 does not extend beyond the bevel 506 (which may interfere with the bevel 506 entering the vein). With the tip 138 retracted into the needle 142, imaging operations may be temporarily suspended until the bevel 506 of the needle 142 is fully disposed within the vein 704. After a brief period, the tip 138 may then reextend beyond the bevel 506 and imaging operations may re-commence to confirm that penetration of the vein 704 is complete.

In at least some implementations, the guidance information displayed by the display device 118 of the control system 110 may change based on whether the medical instrument had made contact with the target tissue. For example, as a result of the needle 142 making contact with the vein 704, the guidance information displayed by the display device 118 of the control system 110 may be adjusted (or augmented). FIG. 12 shows a third view 1200 displayed by the display device 118 of the control system 110 during the medical procedure in accordance with an embodiment of the present disclosure. In this implementation, the third view 130 shows a circle 1202 representing a view along a longitudinal axis of the cylindrical vein 704 (or other target tissue), and a displayed needle bevel 1204 representing the location of the bevel 506 of the needle 142 relative to the vein 704. The guidance information provided by the third view 1200 may inform the medical practitioner of the degree of insertion of the needle 142 into the target vein 704 (represented by circle 1202) and assist in the decision whether the needle 142 has reached the desired position for performing the medical procedure.

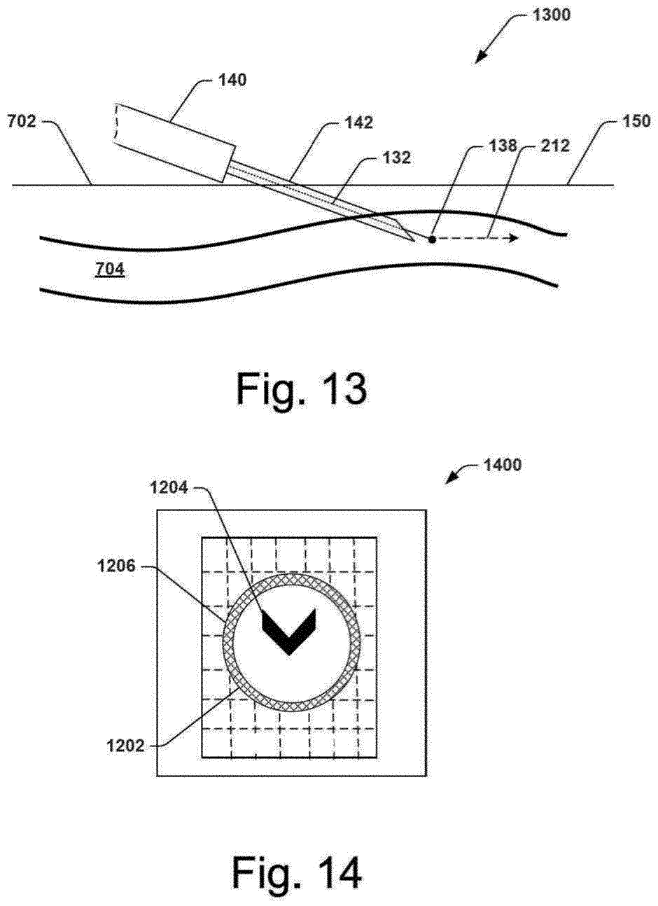

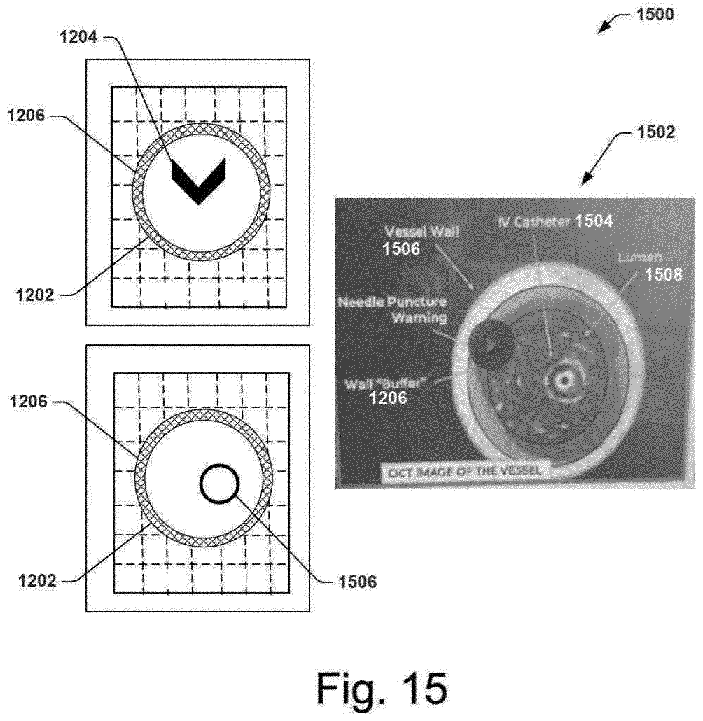

In addition, FIG. 13 shows the needle 142 of the handheld component 140 positioned in a third position 1300 such that the bevel 506 (and bevel tip 504) have fully entered the vein 704 of the body portion 150 of the patient. Similarly, FIG. 14 shows a fourth view 1400 displayed by the display device 118 of the control system 110 during the medical procedure. In this implementation, the fourth view 1400 shows the displayed needle bevel 1204 fully located within the circle 1202 representing the vein 704 (or other target tissue). It will be appreciated that the displayed needle bevel 1204 may change color (e.g. from red to green) once the needle bevel 1204 is fully within the target vein 704, thereby providing an additional visual indication to the medical practitioner that the needle 142 has reached the desired position to perform the medical procedure. In addition, as shown in FIG. 14, a buffer region 1206 may be displayed (e.g. indicated by cross hatching, color, highlighting, etc.) within the interior perimeter of the circle 1202 representing the vein 704 to assist the medical practitioner in properly guiding the needle bevel 1204 to reduce or prevent unintentional contact with the inner surfaces of the vein 704 during the medical procedure.

FIG. 15 shows a fifth view 1500 that may be displayed by the display device 118 of the control system 110 during the medical procedure. In this implementation, the fifth view 1500 shows the displayed needle bevel 1204 fully located within the circle 1202 representing the vein 704 (as described above and shown in FIG. 14). The fifth view 1500 also includes an actual OCT image 1502 that is being acquired by the imaging system 120 during the medical procedure, showing an intravenous (IV) catheter 1504 inserted into the vein 704. Other features visible in the OCT image 1502 include a vessel wall 1506 and an interior lumen 1508 of the vein 704. Also, the buffer region 1206 may be displayed (e.g. indicated by cross hatching, color, highlighting, etc.) within the interior perimeter of the vessel wall 1506 to assist the medical practitioner in properly guiding the medical instrument (i.e. catheter 1504) to reduce or prevent unintentional contact with the vessel wall 1506 during the medical procedure. Finally, the fifth view 1500 also includes a displayed catheter 1506 (at lower left) disposed within the vein 704.

Referring again to FIG. 6, upon determining that the medical instrument has reached the desired final position for performing the medical procedure (at 626), the process 600 proceeds to performing the medical procedure with the medical instrument at the desired position at 628. For example, the performing of the medical procedure (at 628) may include intravenous administration of a medication via the needle 142 into the vein 704. In at least some implementations, the control system 110 may signal the stage assemble 130 to withdraw the tip 138 of the second optical fiber 132 into a retracted position within the needle 142 during (or prior to) the performing of the medical procedure. After the medical procedure is performed (at 628), the process 600 ends or continues to other operations at 630.

Techniques and technologies in accordance with the present disclosure may advantageously provide guidance for medical practitioners for guiding medical instruments during medical procedures using real-time imaging. By employing technologies in accordance with the present disclosure, such guidance information may improve the performance of medical procedures by enabling such procedures to be performed more accurately and with less repetition in comparison with prior art techniques.

The preceding discussion has described various implementations of systems and processes in accordance with the present disclosure. It should be appreciated, however, that in alternate implementations, techniques and technologies in accordance with the present disclosure may involve additional details, operations, or activities than those discussed above. Therefore, the following discussion is intended to provide additional descriptive subject matter that may be employed in various further implementations in accordance with the present disclosure.

As noted above, in at least some implementations, an optical coherence tomography (OCT) probe may be used to provide real-time visual interactive guidance of a medical instrument (such as a needle) toward a target tissue (such as a blood vessel). In some implementations, real-time translation of discrete distance measurements from the OCT probe to the blood vessel, at specific inclinations to the needle axis, may be converted into quantitative information concerning the position, orientation, and size of the blood vessel, so as to provide real-time visual guidance to the user. For example, an OCT probe may be configured to emit a sequence of light pulses along a conical surface with a beam angle φ=60° relative to the needle axis and at equidistant azimuthal spacing δθ=0.5° about the axis, which may yield accurate distance measurements to points on the blood vessel. Several scans may be made, at successive extensions δz of the probe from the needle tip.

In at least some implementations, during analysis of imaging data to determine a cylindrical shape of the target tissue (at operation 616 of FIG. 6), the blood vessel (or at least a local portion thereof) is modeled as a cylindrical surface, and since discrete point data determined by the OCT probe are of finite accuracy, a suitable fitting routine, such as a least-squares approach, may be applied to the point data to identify the cylinder and to suppress possible influence of measurement noise. A cylinder may be identified by its radius, its axis, and a point on the axis. The implicit equation f (x, y, z)=0 expressed in terms of these intuitive parameters, however, has a non-linear dependence on these parameters. The use of an iterative solution procedure may in some cases be incompatible with real-time computation, or may fail to converge when suitable starting approximations cannot be determined a priori.

In at least some implementations, an approach may avoid such problems by performing a least-squares fit of the data points to a general quadric surface, resulting in a linear system of equations for the unknown coefficients. These coefficients may be regarded as the elements of a symmetric 4 by 4 matrix, and an analysis of the eigenvalues and eigenvectors of the symmetric matrix allows a “best” cylinder fit to be identified in a relatively efficient and reliable manner. Additional details of various techniques for fitting quadric surfaces to data points determined during scanning operations is provided below, however, it may be noted that in alternate implementations, quadric fitting is not used to determine the shape of the target tissue.

In the preceeding discussion, the imaging system 120 shown in FIG. 2 was described as an OCT-based imaging system. In at least some implementations, a broad-spectrum light source in the near infrared or infrared band is used to optimize depth of penetration into the tissues of the patient, with the light being transmitted from the end of a fiber optic line. Although a large amount of the light is lost by absorption or scattering, a fraction of the emitted light (e.g. 10⁻⁶ to 10⁻⁹) may be scattered by a tissue feature, and returns back along the fiber to an interferometer that uses coherent detection (constructive/destructive interference of transmitted and returned signals) to obtain image resolution <10 μm over depths of 1-2 mm.

Because the round-trip time-of-flight of the light may be too short to measure accurately, the data are transformed into the distance or the frequency domain. An early OCT implementation, known as Time Domain (TD) OCT, was based on interference of signals from a sample and a reference arm mirror. The need for rapid, accurate, and repeatable mirror movement may undesirably limit the resolution achievable through such a TD OCT. Accordingly, a more recent Fourier Domain (FD) technology known as a Swept Source (SS) OCT may be employed to achieve substantial improvements in signal acquisition rates and signal-to-noise ratios.

In at least some implementations, an SS OCT may utilize a chirped (i.e. rapid wavelength swept) laser light source and Fast Fourier Transform (FFT) analysis to transform the data from amplitude vs. frequency to intensity vs. depth. Alternately, Spectral Domain (SD) OCT (which is another FD OCT variant) may provide substantial improvements in both sampling rates and signal-to-noise ratio over TD OCT.

It should be appreciated that, regardless of method, the OCT system may provide a one-dimensional array of tissue reflectivity as a function of incremental depth. As noted above, such an array may be referred to as an “A-line.” Multiple A-lines can be aggregated as a “B-scan” defining a raster scan in either Cartesian (x, y) or polar (r, θ) coordinates. In addition, in some implementations, A-lines may also be assembled left-to-right as a function of time, yielding a “waterfall” diagram representing reflectivity along a particular vector (light vector 508 of FIG. 5) in the tissue as a function of depth and time. The waterfall format may be useful in studying temporal variations. In the implementation described above, the OCT probe (tip 138) is rotated as a function of time, and angle data is reconstructed from time stamps. The B-scan indices may then be interpreted as polar coordinates to create a “radar” plot.

Various known signal processing methods can be applied to the A-lines or B-scans to generate images of the tissues, or to extract information useful for diagnostic or procedural purposes. For guiding a needle during a medical procedure, for example, the goal is to identify the instantaneous position and orientation of a blood vessel (or other target tissue) relative to the needle tip. In at least some implementations, this may be accomplished by surface reconstruction in B-Scan space. Real-time collection and interpretation of OCT data for navigation and therapeutic purposes, and including methods for steering the needle to the blood vessel. It should be appreciated that contemporary OCT is most frequently used in a post-processing workflow such that the imaging data is collected and then later analyzed off-line for presentation in a non-real-time manner in a diagnostic context.

Importantly, although one or more algorithms for identifying a blood vessel are described herein based on OCT, such algorithms are not necessarily restricted to implementations with OCT-based imaging systems. Any suitable imaging modalities that can reconstruct a list of surface detection events may benefit from the one or more algorithms disclosed herein (assuming adequate resolution).

Additional description of techniques for analyzing imaging data to identify a target tissue (such a blood vessel) may further include a discussion of general quadric surfaces. The data generated by the OCT probe correspond to a discrete sampling of points on an intersection curve of an indeterminate cylinder (the blood vessel) with a known cone of sampling light. In at least some implementations, the problem is to determine the position, orientation, and radius of the cylinder from these data points. Cones and cylinders are special instances of a family of algebraic surfaces of degree 2, commonly known as the quadric surfaces. The characterization of quadric surfaces is a well-known topic in algebraic geometry. The implicit equation of a general quadric may be specified in terms of a symmetric 4×4 matrix through the expression

$$q ( x , y , z ) = [ \begin{matrix}x & y & z & 1 ] [ \begin{matrix}a & f & h & l \\ f & b & g & m \\ h & g & c & n \\ l & m & n & d\end{matrix} ] [ \begin{matrix}x \\ y \\ z \\ 1\end{matrix} ] = 0.\end{matrix}$$

Expanding the matrix product gives the following Equation (1):

ax²+by²+cz²+2fxy+2gyz+2hzx+2lx+2my+2nz+d=0. (1)

The eigenvectors of the upper-left 3×3 sub-matrix

$$[ \begin{matrix}a & f & h \\ f & b & g \\ h & g & c\end{matrix} ] \qquad ( 2 )$$

determine the principal axes of the quadric surface. The eigenvalues are the roots ξ of the characteristic equation

ξ³−βξ²+γξ−δ=0, (3)

with coefficients

β:=a+b+c, γ:=ab+bc+ca−f²−g²−h², (4)

δ:=abc+2fgh−ag²−bh²−cf², (5)

Since the matrix (2) is symmetric, its eigenvalues are all real. The quantities β, γ, δ—together with the determinant

$$Δ := \vert \begin{matrix}a & f & h & l \\ f & b & g & m \\ h & g & c & n \\ l & m & n & d\end{matrix} \vert$$

of the 4×4 matrix—are invariants of the quadric surface, which is to say they remain unchanged under a motion (translation/rotation) of the surface.

Hereinafter, the terms “cylinder” and “cone” will be understood as referring to right circular cylinders and cones, whose sections by any plane orthogonal to their axes is a circle. The cones and cylinders are ruled quadric surfaces, generated by a one-parameter family of lines. For a cone, these lines pass through a fixed point (the vertex) (or the tip 138 described above), and maintain a constant angle with a fixed line (the axis). For a cylinder, the lines are parallel to and equidistant from a fixed line (the axis). A cylinder may be regarded as a special instance of a cone, with a point at infinity as the vertex, and we refer to the set of all cones and cylinders as generalized cones. The generalized cones are singular quadrics, distinguished by the condition Δ=0. In terms of the other invariants, a cone is identified by the condition δ≠0, and a cylinder is identified by δ=0, γ≠0. These conditions identify all (not just right circular) cones and cylinders.

With δ=0≠γ equation (3) reduces, on factoring out the root ξ=0, to

ξ²−βξ+γ=0,

and a right circular cylinder is identified by the condition, β²−4 γ=0, that this quadratic equation should have a double root—namely, ξ=½ β.

A cylinder of general position and orientation may be specified by its radius r, a point p_{*}=(x_{*}, y_{*}, z_{*}) on its axis, and a unit vector a=(λ, μ, ν) satisfying

λ²+μ²+ν²=1, (6)

that defines the axis orientation. The implicit equation of the cylinder may be written explicitly in terms of these geometrical parameters as described below. Specifically, the position of a general point p=(x, y, z) relative to p_{*} can be resolved into components parallel and perpendicular to the axis a as

p−p_{*}=[(p−p_{*})·a]a+a×[(p−p_{*})×a].

The equation of the cylinder is then determined from the condition that the perpendicular distance of p from the axis is r, and this reduces to

|a×(p−p_{*})|²=r². (7)

Equation (7) may be re-expressed in terms of the coordinates of p, and making use of Equation (6), we obtain the implicit equation

$$f ( x , y , z ) = ( 1 - {λ}^{2} ) {( x - {x}_{*} )}^{2} - 2 λμ ( x - {x}_{*} ) ( y - {y}_{*} ) + ( 1 - {μ}^{2} ) {( y - {y}_{*} )}^{2} - 2 μ v ( y - {y}_{*} ) ( z - {z}_{*} ) + ( 1 - {v}^{2} ) {( z - {z}_{*} )}^{2} - 2 v λ ( z - {z}_{*} ) ( x - {x}_{*} ) - {r}^{2} = 0. \qquad ( 8 )$$

Note that this equation is invariant upon replacing (x_{*}, y_{*}, z_{*}) by (x_{*}, y_{*}, z_{*})+α (λ, μ, ν) for any α—i.e., it does not depend on the choice of the point p_{*} on the cylinder axis. In the present context, we may assume that z_{*}=0 (this is valid if ν≠0, i.e., the cylinder axis is not parallel to the (x, y) plane).

The form (8) corresponds to coefficients in the general quadric equation specified by

(a,b,c)=(1−λ²,1−μ²,1−ν²,(f,g,h)=(−λμ,−μν,−νλ), (9)

(l,m,n)=(−ax_{*}−fy_{*}−hz_{*},−fx_{*}−by_{*}−gz_{*},hx_{*}−gy_{*}−cz_{*}), (10)

d=ax_{*}²+by_{*}²+cz_{*}²+2fx_{*}y_{*}+2gy_{*}z_{*}+2hz_{*}x_{*}−r², (11)

where it is understood that the constraint (6) also holds.

In principle, a quadric surface can be uniquely determined from 9 points lying in “general position” on it, since equation (1) depends on 10 coefficients, and the surface is unchanged upon dividing (1) by any non-zero coefficient. However, since the 9 points must be exactly specified, and verifying that they are in “general position” is non-trivial, this approach is impractical.

In at least some implementations, given N data points pᵢ=(xᵢ, yᵢ, zᵢ), i=1, . . . , N on the intersection of a known cone and a cylinder, we wish to determine the cylinder. Since the data will be subject to measurement noise, a least-squares fitting scheme is desirable to suppress the influence of the noise. The least-squares fit may be based on either the general quadric surface equation (1), or the equation (8) expressed in terms of the cylinder geometrical parameters.

Equation (8) explicitly determines a cylinder in terms of the geometrical parameters p_{*}, a, r. However, the dependence upon these parameters is not linear, and the least-squares fit will incur a constrained system of non-linear equations. A computationally-intensive iterative method is required to solve this system, and without a reliable scheme for choosing “good” starting values it will not be sufficiently robust for real-time implementation.

Equation (1), on the other hand, is linear in the coefficients a, b, c, . . . and the least-squares fit incurs a system of linear equations for these unknowns, that has a unique solution (if the matrix defined by equations (13)-(15) below is non-singular). Since the general quadric equation (1) does not explicitly determine the least-squares fit surface as a cylinder, the geometry parameters p_{*}, a, r of the “nearest” true cylinder must be extracted from the computed coefficients a, b, c, . . . , as described in Section 6.

In view of the above considerations, equation (1) will be employed in the least-squares surface fit. As observed above, the OCT scan identifies points on the intersection curve of a known cone with the unknown cylinder. This amounts to a one-dimensional sampling of a two-dimensional surface that is, in general, insufficient to uniquely identify the surface. Two or more scans, at different extensions δz of the probe along the needle axis, are required.

This may be seen as follows. The intersection of two quadric surfaces q₀(x, y, z)=0 and q₁(x, y, z)=0 is, in general, an irreducible quartic space curve, which is to say it may degenerate into a collection of simpler curves (lines, conics, and cubics) whose degrees sum to 4. There are infinitely many pairs of quadric surfaces that possess the same intersection curve C as q0(x, y, z)=0 and q1(x, y, z)=0. Any two members of the pencil of quadrics defined by

q^{τ}(x,y,z)=(1−τ)q₀(x,y,z)+τq₁(x,y,z)=0, −∞<τ<+∞

corresponding to distinct τ values possess the same intersection curve C as q₀(x, y, z)=0 and q₁(x, y, z)=0. Thus, given one of two quadrics, it is not possible to uniquely identify the other from their intersection curve.

In the present context, one quadric is a known cone, and we can exploit the additional information that the unknown quadric is a cylinder. Suppose Q₀ and Q₁ are symmetric 4×4 matrices with elements a₀, b₀, . . . and a₁, b₁, . . . , specifying two quadric surfaces. Then the determinantal equation

p(ρ)=|(1−τ)Q₀+τQ₁|=0

is of degree 4, and its (real) roots identify the generalized cones of the pencil defined by Q0 and Q1. The quartic polynomial p(τ) is called the discriminant of the pencil of quadrics. In the generic case, in which the roots of p(τ) are distinct, the intersection C is a non-singular quartic space curve.

To verify that a cylinder Q₁ constructed from a known intersection curve C with a known cone Q₀ is unique, we must determine the real roots of the discriminant p(τ) of the pencil defined by Q₀ and Q₁, and check that none of the quadrics corresponding to these roots (other than τ=1) is a cylinder. One known method (known as Ferrari's method) provides a closed-form solution for all the roots of p(τ). This uniqueness test can only be performed a posteriori, i.e., after Q1 has been constructed. However, using multiple scans at successive extensions δz of the OCT probe eliminates the need to perform this test.

Equation (1) may be divided by any non-zero coefficient without influencing the quadric surface it defines. In the present context, we may divide through by d, which corresponds to the choice d=1 in (1). This is permissible if the surface q(x, y, z)=0 does not pass through the origin, which is true since the origin is defined to be the apex of the cone (i.e., the position of the sensor) and the sensor does not encroach on the cylinder.

In at least some implementations, a coordinate system is adopted in which the needle axis is identified with the z-axis, and for zero extension the OCT probe is located at z=0. The known parameters and available data are the cone beam angle φ (deflection angle φ of the light vector 508) about the z-axis, the measured distances ρᵢ from the probe to the blood vessel surface, and the associated azimuthal angles θᵢ on the cone and probe extensions δzᵢ for each measured point, i=1, . . . , N. For the least-squares fit, the data are converted to Cartesian coordinates according to

xᵢ=ρᵢ sin φ sin θᵢ, yᵢ=ρᵢ sin φ cos θᵢ, zᵢ=ρᵢ cos φ+δzᵢ. (12)

With d=1, the remaining unknown 9 coefficients a, b, c, f, g, h, l, m, n in (1) are determined by minimizing the expression

$$E = {\sum }_{i = 1}^{N} {q}^{2} ( {x}_{i} , {y}_{i} , {z}_{i} ) .$$

Setting the partial derivatives of E with respect to these coefficients equal to zero results in a linear system of equations of the form

Mv=r, (13)

where v=[a b c f g h l m n]^{T} and, on introducing the basis functions

ϕ₁(x,y,z)=x², ϕ₂(x,y,z)=y², ϕ₃(x,y,z)=z²,

ϕ₄(x,y,z)=2xy, ϕ₅(x,y,z)=2yz, ϕ₆(x,y,z)=2zx,

ϕ₇(x,y,z)=2x, ϕ₈(x,y,z)=2y, ϕ₉(x,y,z)=2z, (14)

the elements of the matrix M and right-hand side vector r can be expressed in terms of the data points (xᵢ, yᵢ, zᵢ) as

$$\begin{matrix}{M}_{jk} = {\sum }_{i = 1}^{N} {ϕ}_{j} ( {x}_{i} , {y}_{i} , {z}_{i} ) {ϕ}_{k} ( {x}_{i} , {y}_{i} , {z}_{i} ) , & 1 \leq j , k \leq 9 ,\end{matrix} \qquad ( 15 )$$

$$\begin{matrix}{r}_{j} = - {\sum }_{i = 1}^{N} {ϕ}_{j} ( {x}_{i} , {y}_{i} , {z}_{i} ) , & 1 \leq j \leq 9.\end{matrix} \qquad ( 16 )$$

The linear system (13) has a unique solution when M is non-singular, which can be efficiently computed by Gaussian elimination.

Techniques for determining the cylinder geometry parameters will now be described. Once the vales a, b, c, f, g, h, l, m, n have been computed, we must obtain the cylinder geometrical parameters p_{*}, a, r from them. The principal axes of the quadric surface are identified by the eigenvectors (vₓ, v_{y}, v_{z}) of the 3×3 matrix (2)—i.e., by the solutions of the equation

$$[ \begin{matrix}a - ξ & f & h \\ f & b - ξ & g \\ h & g & c - ξ\end{matrix} ] [ \begin{matrix}{υ}_{x} \\ {υ}_{y} \\ {υ}_{z}\end{matrix} ] = [ \begin{matrix}0 \\ 0 \\ 0\end{matrix} ] , \qquad ( 17 )$$

where the eigenvalues ξ are the roots of the characteristic equation (3) with the coefficients (4)-(5). As noted above, for an exact right circular cylinder ξ=0 is one eigenvalue (with no valid associated eigenvector), and ξ=2 β is a double eigenvalue, with which we may associate two linearly-independent eigenvectors. The latter eigenvectors span a diametral plane of the cylinder, orthogonal to its axis. Hence, the three row vectors of the 3×3 matrix in (17) must be parallel (or anti-parallel) to the cylinder axis.

If the coefficients a, b, c, f, g, h are determined from a least-squares fit to noisy data, they will not exactly define a right circular cylinder, and the row vectors of the 3×3 matrix in (17) will not be precisely parallel or antiparallel. To estimate the cylinder axis, we form the three unit vectors

$$\begin{matrix}{u}_{1} = \frac{( a - ξ , f , h )}{\vert ( a - ξ , f , h ) \vert} , & {u}_{2} = \frac{( f , b - ξ , g )}{\vert ( f , b - ξ , g ) \vert}\end{matrix} , \qquad {u}_{3} = \frac{( h , g , c - ξ )}{\vert ( h , g , c - ξ ) \vert}$$

taking u₁ as a reference, we reverse u₂ if u₁·u₂<0 and u₃ if u₁·u₃<0. The cylinder axis a is then estimated as the centroid of these unit vectors, namely

$$a = \frac{{u}_{1} + {u}_{2} + {u}_{3}}{\vert {u}_{1} + {u}_{2} + {u}_{3} \vert} . \qquad ( 18 )$$

Consider next the determination of the point p_{*}=(x_{*}, y_{*}, z_{*}) on the axis. As previously noted, we may assume that z_{*}=0 if the cylinder axis is not parallel to the (x, y) plane. With d=1, the restriction of (1) to the plane z=0 identifies a conic curve specified by the equation

ax²+by²++2fxy+2lx+2my+1=0. (19)

Provided that ab−f²≠0, this defines a central conic, and its center identifies the intersection of the cylinder axis with the (x, y) plane. The center can be determined by identifying the shift (x, y) (x+x_{*}, y+y_{*}) of the origin that will eliminate the terms of (19) linear in x and y. One can easily verify that

$$( {x}_{*} , {y}_{*} ) = ( \frac{fm - l b}{ab - {f}^{2}} \cdot \frac{fl - ma}{ab - {f}^{2}} ) . \qquad ( 20 )$$

The final parameter to be determined is the cylinder radius r. Knowing the cylinder axis a and a point p* on it, a robust approach is to compute r as the root-mean-square distance of the N data points pi=(xi, yi, zi) from the cylinder axis. Thus, based on equation (7), the radius is estimated as

$$r = {[ \frac{1}{N} {\sum }_{i = 1}^{N} {\vert ( {p}_{i} - {p}_{*} ) \times a \vert}^{2} ]}^{1 / 2} . \qquad ( 21 )$$

For a quadric surface defined by equation (1) that is a true right circular cylinder, and exact data points p₁, . . . , p_{N}, the above procedure can precisely identify its geometry parameters. First, with the eigenvalue ξ=½ (a+b+c), the rows of the of the 3×3 matrix in (17) will be precisely linearly dependent, and unitizing any of them will exactly determine the axis vector a. Moreover, the point p_{*}=(x_{*}, y_{*}, z_{*}) on the axis with z_{*}=0 is precisely identified by (20). Finally, any exact point pᵢ on the cylinder will suffice to determine the radius as r=|(pᵢ−p_{*})×a|.

Solution reliability of the foregoing technologies and algorithms will now be discussed. For the vector norm

∥v∥ₚ=(|a|^{p}+ . . . +|n|^{p})^{1/p},

the subordinate norm of the 9×9 matrix M in (13) may be specified as

$${‖ M ‖}_{p} = {max}_{v \neq 0} \frac{{‖ Mv ‖}_{p}}{{‖ v ‖}_{p}} ,$$

and the p-norm condition number Cₚ(M) of M is defined by

Cₚ(M)=∥M∥ₚ∥M⁻¹∥^{p}.

If a perturbation δr is imposed on the right-hand-side vector r in (13), that incurs a corresponding perturbation δv in the solution vector v, the relative errors

ϵᵥ=∥δv∥ₚ/∥v∥ₚ

and

ϵᵣ=∥δr∥ₚ/∥r∥ₚ

satisfy

ϵᵥ≤Cₚ(M)ϵᵣ. (22)

The bound (22) is sharp, i.e., it holds with equality for some perturbation δr. In the cases p=1 and ∞, ∥M∥ₚ is the greatest of the column and row sums of absolute values of the matrix elements, respectively. Since M and M⁻¹ are symmetric, ∥M∥₁=∥M∥_{∞}, ∥M⁻¹∥₁=∥M⁻¹∥_{∞}, so C₁(M)=C_{∞}(M), and we may simply write C(M). The condition number gives a (worst-case) indication of the influence of round-off error amplification when the system (13) is solved using floating-point arithmetic.

In the present context, a different source of inaccuracy may be dominant when solving (13). Namely, the elements (15) and (16) of both the matrix M and right-hand side vector r are not known exactly, since they are computed from the basis functions (14) evaluated at the data points (xᵢ, yᵢ, zᵢ), whose precision is limited by the accuracy of the OCT distance measurements ρᵢ.

To assess the influence of the finite accuracy of the distances ρᵢ, they are assumed to have Gaussian (normal) distributions [14] of the form

$$f ( {ρ}_{i} ) = \frac{1}{\sqrt{2 π} σ} exp [ - \frac{{( {ρ}_{i} - {\overline{ρ}}_{i} )}^{2}}{2 {σ}^{2}} ] , \qquad ( 23 )$$

where it is assumed that the nominal distance measurements are reasonable estimates of their individual means ρ̅ᵢ, and the same standard deviation σ=0.0005 mm holds for each measurement—this corresponds to ˜68% of the measured distances ρᵢ being within ±0.0005 mm of ρ̅ᵢ. In at least some implementations, a Monte Carlo experiment may be performed in which each individual ρᵢ is randomly perturbed to a new value \tilde{ρ}ᵢ in accordance with the probability distribution (23). New point coordinates (\tilde{x}ᵢ, \tilde{y}ᵢ, \tilde{z}ᵢ) are then computed from the \tilde{ρ}ᵢ values using (12), and the corresponding matrix elements \tilde{M}ⱼₖ and right-hand-side values \tilde{r}ⱼ are obtained from (15) and (16). Solving the resulting linear system \tilde{M}\tilde{v}=\tilde{r}, for the resulting perturbed coefficients \tilde{v}=[ã \tilde{b} \tilde{c} \tilde{f} \tilde{g} \tilde{h} \tilde{l} \tilde{m} ñ]^{T}, we define their relative error as

$${ϵ}_{v} = \frac{{‖ \tilde{v} - v ‖}_{2}}{{‖ v ‖}_{2}} .$$

The Monte Carlo experiment may be repeated several times, with different random samplings of the distributions (23), to assess the overall consistency and range of variation in the ϵᵥ values obtained. Examples described below confirm that this approach offers a favorable assessment of the accuracy of the computed quadric surface coefficients.

Three examples will now be described that demonstrate the effectiveness of implementations of the above-described techniques and technologies in accordance with the present disclosure. More specifically, the following examples describe results obtained from an implementations of the methodology in the C programming language on representative test data sets (all dimensions are in mm). In the conversion (12) of the “raw” OCT probe data to Cartesian coordinates, the cone beam angle (deflection angle of light vector 508 of FIG. 5) is φ=60° and the scans are made at azimuthal angle increments δθ=0.5° for each fixed probe extension δz.

In a first representative example in accordance with the present disclosure, the cylinder has radius r=0.75, and the axis is specified by the point p_{*}=(x_{*}, y_{*}, z_{*})=(1.0, 4.0, 0.0) and the unit vector a=(λ, μ, ν)=(−0.17364818, −0.33682409, 0.92541658). Scans are made at three successive extensions δz, the distances ρ to the cylinder being detected at the angular increment δθ beginning at θ₀, for a total of n points per scan as follows:
δ_{z}=0.0, θ₀=−2.0°, n=52;
δ_{z}=1.0, θ₀=−5.0°, n=57;
δ_{z}=2.0, θ₀=−9.0°, n=65.

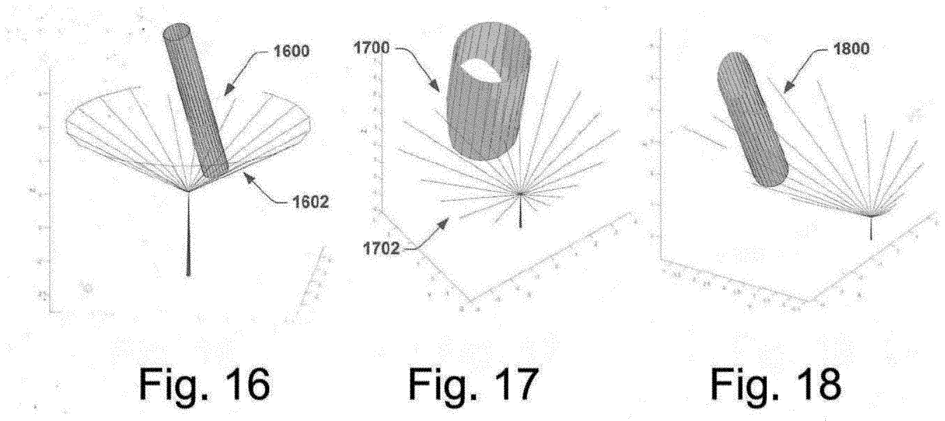

The total number of points is N=174. Table 1 compares the exact cylinder coefficients, computed from (9)-(11) and divided by d, with the least-squares fit values. From (18) we obtain a=(−0.17503840, −0.33666708, 0.92521178) as the estimated cylinder axis, which makes an angle 0.081015° with the exact axis (−0.17364818, −0.33682409, 0.92541658). The axis point p_{*}, determined from (20) has coordinates (x_{*}, y_{*})=(1.00064338, 4.00260039), as compared to the exact point (1.0, 4.0). Finally, the cylinder radius computed from (21) is r=0.746531, whereas the exact value is r=0.750000. From the computed coefficients we have values γ=0.00499410, δ=0.00000022 of the invariants (4), in relatively good agreement with the conditions γ=0=δ identifying a cylinder. FIG. 16 shows a first computed cylinder 1600 based on a first scanning pattern 1602 in accordance with the first example simulation described above.

TABLE 1
Comparison of exact and least-squares
fit coefficients for Example 1.

| | exact | least-squares |
|---|---|---|
| a | 0.06866544 | 0.06847202 |
| b | 0.06276800 | 0.06266601 |
| c | 0.01016722 | 0.01015567 |
| f | −0.00414103 | −0.00413862 |
| g | 0.02206865 | 0.02197726 |
| h | 0.01137739 | 0.01133276 |
| l | −0.05210131 | −0.05195083 |
| m | −0.24693097 | −0.24668571 |
| n | −0.09965198 | −0.09946885 |
| d | 1.00000000 | 1.00000000 |

The condition number of the matrix M in this example is C(M)=1.81×10⁶. The Monte Carlo accuracy assessment (described in Section 7) was run 100 times with different random numbers satisfying the Gaussian distribution (23), resulting in values of the fractional error ϵᵥ in the computed coefficients ranging between 0.000096 and 0.001095, with a mean value 0.000508.

Overall, the least-squares fitting procedure and the parameter estimation scheme discussed above provide a remarkably accurate estimation of the cylinder geometry, despite the relatively low precision of the measurement data. To demonstrate that the accuracy of the data is the only factor limiting the precision with which the cylinder can be identified, the computation was repeated with ρ values computed in double-precision arithmetic, in lieu of the values with 3 decimal place accuracy used above. This resulted in an angular deviation between the estimated and exact axes of only 0.0000008538°, and (x_{*}, y_{*})=(1.000000000029, 4.000000000119), r=0.749999999795 for the coordinates of the axis point p_{*} and the cylinder radius r.

In a second representative example in accordance with the present disclosure, the cylinder geometry parameters were set to (x_{*}, y_{*})=(1.0, 4.0), a=(0.64278761, 0.26200263, 0.71984631), and r=1.5. Three scans were made, corresponding to the values
δ_{z}=0.0, θ₀=−15.5°, n=106;
δ_{z}=1.0, θ₀=−20.5°, n=119;
δ_{z}=2.0, θ₀=−26.5°, n=134.

The total number of points is N=359. Table 2 compares the exact cylinder coefficients, computed from (9)-(11) and divided by d, with the least-squares fit values. From (18) we obtain a=(−0.18374332, −0.32542690, 0.92754284) as the estimated cylinder axis, which makes an angle 0.880816° with the exact axis (−0.17364818, −0.33682409, 0.92541658). The axis point p_{*}, determined from (20) has coordinates (x_{*}, y_{*})=(0.99717264, 3.98746160), as compared to the exact point (1.0, 4.0). Finally, the cylinder radius computed from (21) is r=1.522544, whereas the exact value is r=1.500000. From the computed coefficients we have values γ=0.00641154, δ=0.00000007 of the invariants (4), as compared to the exact conditions γ≠0=δ defining a cylinder. FIG. 17 shows a second computed cylinder 1700 based on a second scanning pattern 1702 in accordance with the first example simulation described above.

The condition number of the least-squares matrix in this case is C(M)=1.65×10⁵. The Monte Carlo accuracy assessment was run 100 times with different random numbers satisfying the Gaussian distribution (23), yielding values of the fractional error ϵᵥ in the computed coefficients between 0.000068 and 0.000738, with a mean value 0.000303.

When the computation is repeated with double-precision ρ values, in lieu of the values with 3 decimal place accuracy used above, we obtain an angular deviation between the estimated and exact axes of 0.0000000000°, and (x_{*}, y_{*})=(0.999999999997, 3.999999999988), r=1.500000000018 for the coordinates of the axis point p_{*} and the cylinder radius r.

TABLE 2
Comparison of exact and least-squares
fit coefficients for Example 2.

| | exact | least-squares |
|---|---|---|
| a | 0.07798243 | 0.07709279 |
| b | 0.07128479 | 0.07145285 |
| c | 0.01154678 | 0.01150307 |
| f | −0.00470292 | −0.00441349 |
| g | 0.02506307 | 0.02517422 |
| h | 0.01292116 | 0.01287786 |
| l | −0.05917077 | −0.05927620 |
| m | −0.28043625 | −0.28051450 |
| n | −0.11317344 | −0.11327718 |
| d | 1.00000000 | 1.00000000 |

In a third representative example in accordance with the present disclosure, the cylinder geometry parameter are (x_{*}, y_{*})=(1.0, 4.0), a=(0.64278761, 0.26200263, 0.71984631), and r=0.5. Three scans were made, corresponding to the values
δ_{z}=0.0, θ₀=−24.5°, n=48;
δ_{z}=1.0, θ₀=−42.0°, n=53;
δ_{z}=2.0, θ₀=−59.0°, n=52.

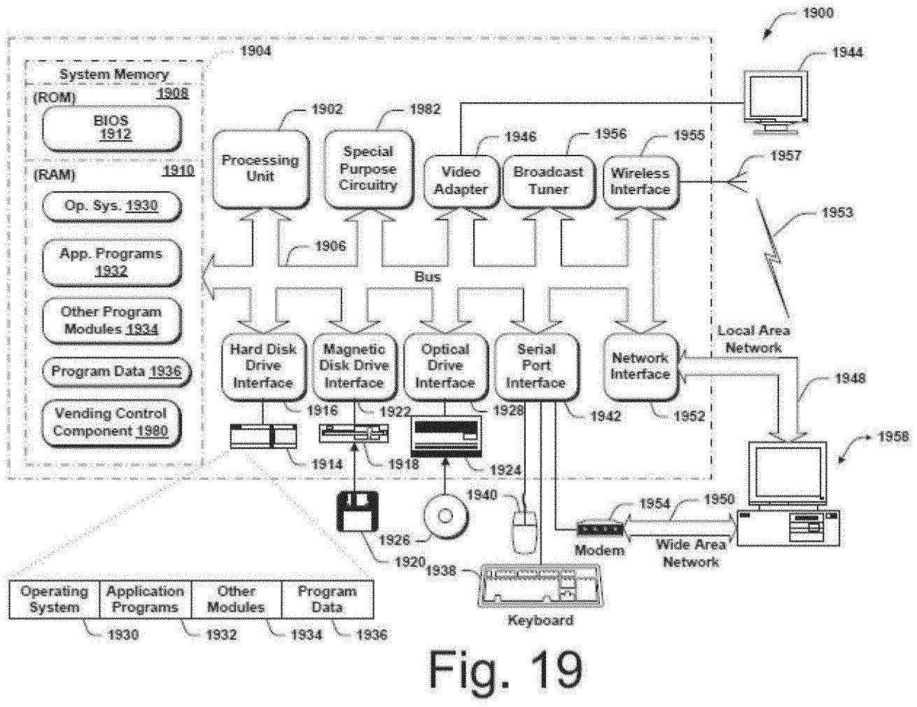

The total number of points is N=153. Table 3 compares the exact cylinder coefficients, computed from (9)-(11) and divided by d, with the least-squares fit values. From (18) we obtain a=(−0.64386222, −0.25900228, 0.71997171) as the estimated cylinder axis, which makes an angle 0.182742° with the exact axis (−0.64278761, −0.26200263, 0.71984631). The axis point p_{*}, determined from (20) has coordinates (x_{*}, y_{*})=(1.00228517, 4.00163039), as compared to the exact point (1.0, 4.0). Finally, the cylinder radius computed from (21) is r=0.507851, whereas the exact value is r=0.500000. From the computed coefficients we have values γ=0.00514512, δ=0.00000002 of the invariants (4), in fair agreement with the conditions γ=0=6 characterizing a cylinder. FIG. 18 shows a third computed cylinder 1800 based on a third scanning pattern 1802 in accordance with the first example simulation described above.

TABLE 3
Comparison of exact and least-squares
fit coefficients for Example 3.

| | exact | least-squares |
|---|---|---|
| a | 0.04224430 | 0.04195657 |
| b | 0.06704637 | 0.06700938 |
| c | 0.03468536 | 0.03449726 |
| f | −0.01212365 | −0.01213370 |
| g | 0.01357706 | 0.01356661 |
| h | 0.03330945 | 0.03307726 |
| l | 0.00625029 | 0.00650214 |
| m | −0.25606182 | −0.25598535 |
| n | −0.08761768 | −0.08742876 |
| d | 1.00000000 | 1.00000000 |

The condition number of the least-squares matrix M in this example is C(M)=1.20×10⁷. Using 100 runs of the Monte Carlo accuracy assessment with different random numbers that satisfy the Gaussian distribution (23), values of the fractional error Ev in the computed coefficients between 0.000110 and 0.001457 were obtained, with a mean value 0.000557. As in the preceding examples, essentially exact cylinder geometry parameters were obtained when the computation was repeated with double-precision ρ values.

The suitability of the techniques and technologies described herein for real-time implementation will now be described. In at least some implementations, using a modest 1.1 GHz processor, the execution times for identification of the cylinder from the point coordinate data in the three examples described above were 0.27 ms, 405 ms, and 0.24 ms, respectively. Since these examples used N=174, 359, and 153 points, these execution times are consistent with a linear dependence on N, and constitute only a modest fraction of the overall effort required for real-time implementation.

In at least some implementations, the OCT probe tip (tip 138) may emit light pulses of 40 μs duration every 50 μs, inclined at 60° to the probe axis (axis 144 of FIG. 5). Within the viewing range, reflection intensity data may be acquired along each pulse, up to a few mm from the probe tip. The probe may rotate along its axis at a 10 Hz rate, and its tip may execute a reciprocating motion along the probe axis at a speed 10 mm/s. These motions result in a helical scanning pattern on the target surface.

In at least some implementations, tor a signal of width 30° the probe may require just under 10 ms to trace a scan curve, with sequential scans at 100 ms apart. Further computations may be needed to convert the raw OCT data into point coordinates, and a target computation time of 100-200 ms per image frame is anticipated. A “rolling” solution to frame updating may also be used, in which overlapping sequences of scans are used to provide a higher image refresh frequency.

Accordingly, techniques and technologies in accordance with the present disclosure may advantageously perform real-time identification of the position, orientation, and size of blood vessels or other desirable target tissues, based on discrete distance measurements from an imaging apparatus. In at least some implementations, the techniques and technologies disclosed herein are sufficiently fast and robust to provide real-time guidance to medical practitioners during medical procedures (e.g. providing needle guidance for venipuncture procedures) through a visual display.

Moreover, modelling a blood vessel (or other target tissues) as a right circular cylinder, techniques and technologies in accordance with the present disclosure may first perform a least-squares fit to the imaging data (e.g. OCT data), in terms of a general quadric surface represented by a symmetric 4×4 matrix. Analysis of the structure of this matrix then allows the right circular cylinder “closest” to the general quadric to be identified. This avoids the need for iterative non-linear surface fitting, which can be computationally demanding, and lacks robustness when identification of a good starting approximation is not available.

Examples described above show that the cylinder identification procedure in accordance with the present disclosure is fast, with a computing time that grows only linearly with the total number N of data points, and the cylinder geometry parameters may be identified with a high degree of robustness. The techniques and technologies in accordance with the present disclosure may therefore be readily adaptable to identification of other simple morphologies, such as general quadrics or toroidal surfaces. Accordingly, techniques and technologies in accordance with the present disclosure may offer significant advantages to medical practitioners for guiding medical instruments during medical procedures using real-time imaging technologies.

In some implementations, one or more aspects of the above-described processes for guiding medical instruments during medical procedures using real-time imaging technologies may be at least partially implemented using a computing device (e.g. the control system 110, etc.). For example, FIG. 19 is a schematic view of an exemplary computing device 1900 configured to operate in accordance with implementations of the present disclosure. As described below, the computing device 1900 can be configured to perform one or more of the functions and operations associated with one or more of the techniques and technologies disclosed herein.

As shown in FIG. 19, in some implementations, the computing device 1900 may include one or more processors (or processing units) 1902, special purpose circuitry 1982, a memory 1904, and a bus 1906 that couples various system components, including the memory 1904, to the one or more processors 1902 and special purpose circuitry 1982. The bus 1906 represents one or more of any of several types of bus structures, including a memory bus or memory controller, a peripheral bus, an accelerated graphics port, and a processor or local bus using any of a variety of bus architectures. In this implementation, the memory 1904 includes read only memory (ROM) 1908 and random access memory (RAM) 1910. A basic input/output system (BIOS) 1912, containing the basic routines that help to transfer information between elements within the computing device 1900, such as during start-up, is stored in ROM 1908.

The exemplary computing device 1900 further includes a hard disk drive 1914 for reading from and writing to a hard disk (not shown), and is connected to the bus 1906 via a hard disk drive interface 1916 (e.g., a SCSI, ATA, or other type of interface). A magnetic disk drive 1918 for reading from and writing to a removable magnetic disk 1920, is connected to the system bus 1906 via a magnetic disk drive interface 1922. Similarly, an optical disk drive 1924 for reading from or writing to a removable optical disk 1926 such as a CD ROM, DVD, or other optical media, connected to the bus 1906 via an optical drive interface 1928. The drives and their associated computer-readable media provide nonvolatile storage of computer readable instructions, data structures, program modules and other data for the computing device 1900. Although the exemplary computing device 1900 described herein employs a hard disk, a removable magnetic disk 1920 and a removable optical disk 1926, it should be appreciated by those skilled in the art that other types of computer readable media which can store data that is accessible by a computer, such as magnetic cassettes, flash memory cards, digital video disks, random access memories (RAMs) read only memories (ROM), and the like, may also be used.

As further shown in FIG. 19, a number of program modules may be stored on the memory 1904 (e.g. the ROM 1908 or the RAM 1910) including an operating system 1930, one or more application programs 1932, other program modules 1934, and program data 1936. Alternately, these program modules may be stored on other computer-readable media, including the hard disk, the magnetic disk 1920, or the optical disk 1926. For purposes of illustration, programs and other executable program components, such as the operating system 1930, are illustrated in FIG. 19 as discrete blocks, although it is recognized that such programs and components reside at various times in different storage components of the computing device 1900, and may be executed by the processor(s) 1902 or the special purpose circuitry 1982 of the computing device 1900.

A user may enter commands and information into the computing device 1900 through input devices such as a keyboard 1938 and a pointing device 1940. Other input devices (not shown) may include a microphone, joystick, game pad, satellite dish, scanner, or the like. These and other input devices are connected to the processing unit 1902 and special purpose circuitry 1982 through an interface 1942 that is coupled to the system bus 1906. A monitor 1944 or other type of display device is also connected to the bus 1906 via an interface, such as a video adapter 1946. In addition to the monitor, the computing device 1900 may also include other peripheral output devices (not shown) such as speakers and printers.

The computing device 1900 may operate in a networked environment using logical connections to one or more remote computers (or servers) 1958. Such remote computers (or servers) 1958 may be a personal computer, a server, a router, a network PC, a peer device or other common network node, (or the automated microscopy assembly 110 of FIG. 1) and may include many or all of the elements described above relative to computing device 1900. The logical connections depicted in FIG. 19 may include one or more of a local area network (LAN) 1948 and a wide area network (WAN) 1950. Such networking environments are commonplace in offices, enterprise-wide computer networks, intranets, and the Internet. In this embodiment, the computing device 1900 also includes one or more broadcast tuners 1956. The broadcast tuner 1956 may receive broadcast signals directly (e.g., analog or digital cable transmissions fed directly into the tuner 1956) or via a reception device (e.g., via an antenna, a satellite dish, etc.).

When used in a LAN networking environment, the computing device 1900 may be connected to the local network 1948 through a network interface (or adapter) 1952. When used in a WAN networking environment, the computing device 1900 typically includes a modem 1954 or other means for establishing communications over the wide area network 1950, such as the Internet. The modem 1954, which may be internal or external, may be connected to the bus 1906 via the serial port interface 1942. Similarly, the computing device 1900 may exchange (send or receive) wireless signals 1953 with one or more remote computers (or servers) 1958, (or with the automated microscopy assembly 110 of FIG. 1), using a wireless interface 1955 coupled to a wireless communicator 1957 (e.g., an antenna, a satellite dish, a transmitter, a receiver, a transceiver, a photoreceptor, a photodiode, an emitter, a receptor, etc.).

In a networked environment, program modules depicted relative to the computing device 1900, or portions thereof, may be stored in the memory 1904, or in a remote memory storage device. The program modules may be implemented using software, hardware, firmware, or any suitable combinations thereof. In cooperation with the other components of the computing device 1900, such as the processing unit 1902 or the special purpose circuitry 1982, the program modules may be operable to perform one or more implementations or aspects of processes in accordance with the present disclosure.

Generally, application programs and program modules executed on the computing device 1900 may include routines, programs, objects, components, data structures, etc., for performing any tasks necessary for the successful implementation of the techniques and technologies for guiding medical instruments during medical procedures using real-time imaging technologies in accordance with the present disclosure. These program modules and the like may be executed as native code or may be downloaded and executed, such as in a virtual machine or other just-in-time compilation execution environments. Typically, the functionality of the program modules may be combined or distributed as desired in various implementations.

In view of the disclosure of techniques and technologies for guiding medical instruments during medical procedures using real-time imaging technologies as disclosed herein, a few representative embodiments are summarized below. It should be appreciated that the representative embodiments described herein are not intended to be exhaustive of all possible embodiments, and that additional embodiments may be readily conceived from the disclosure of techniques and technologies provided herein.

For example, in at least some implementations, a method for performing a medical procedure includes engaging a medical instrument with a body portion of a patient, the medical instrument including an elongated portion configured to be inserted into the body portion and having an optical fiber at least partially disposed within the elongated portion, the optical fiber having a tip portion that is extendable beyond a distal end of the elongated portion; actuating an imaging system that provides a sampling energy into the optical fiber, the sampling energy being emitted from the tip portion along a sampling vector at a deflection angle with the longitudinal axis into one or more tissues of the body portion as the elongated portion is inserted into the body portion, the one or more tissues including a target tissue; and actuating a stage assembly to move the tip portion to perform a scanning of the one or more tissues with the sampling energy emitted from the tip portion, the stage assembly rotating at least the tip portion about a scanning axis that is parallel with the longitudinal axis and reciprocating at least the tip portion along the scanning axis that is parallel with the longitudinal axis with the tip portion proximate to and extended beyond the distal end of the elongated portion, the sampling energy being emitted from the tip portion in a scanning pattern. The method further includes receiving a reflected energy that is reflected from the one or more tissues back through the optical fiber to the imaging system, the reflected energy providing a plurality of one-dimensional arrays of intensity values of the reflected energy at a plurality of distances along the sampling vector; analyzing the plurality of one-dimensional arrays of intensity values of the reflected energy to determine a shape of the target tissue and a location of the target tissue relative to the elongated portion of the medical instrument; and displaying information including one or more relative positions of the target tissue relative to the elongated portion of the medical instrument for guiding the medical instrument into engagement with the target tissue as the elongated portion is inserted into the body portion for performance of the medical procedure.

In further implementations, the method further comprises advancing the distal end of the elongated portion toward the target tissue; and wherein the receiving of the reflected energy, the analyzing of the reflected energy, and the displaying of the information for guiding the medical instrument into engagement with the target tissue are continuously performed in approximately real-time during the advancing of the distal end of the elongated portion toward the target tissue.

In at least some implementations, the analyzing the plurality of one-dimensional arrays of intensity values of the reflected energy to determine a shape of a target tissue and a location of the target tissue relative to the elongated portion of the medical instrument comprises approximating a shape of the target tissue as a cylinder, including determining an axis, a radius, and at least one point on the axis of the cylinder. In still other implementations, the analyzing the plurality of one-dimensional arrays of intensity values of the reflected energy to determine a shape of a target tissue and a location of the target tissue relative to the elongated portion of the medical instrument comprises determining a shape of the target tissue as a quadric surface and performing a least-squares fit of the intensity values onto the quadric surface.

Moreover, in at least some implementations, the scanning pattern includes a conical scanning pattern, and wherein analyzing the plurality of one-dimensional arrays of intensity values comprises analyzing the plurality of one-dimensional arrays of intensity values to determine an intersection of a cylindrical target tissue with the conical scanning pattern. In further implementations, the scanning pattern includes a helical scanning pattern, and wherein analyzing the plurality of one-dimensional arrays of intensity values comprises analyzing the plurality of one-dimensional arrays of intensity values to determine an intersection of a cylindrical target tissue with the helical scanning pattern.

In further implementations, the method further comprises, when the distal end of the elongated portion contacts the target tissue, retracting the tip portion of the optical fiber into the distal end and at least temporarily suspending at least the reciprocating of the tip portion along the longitudinal axis until the distal end of the elongated portion penetrates an outer wall of the target tissue.

In at least some implementations, the imaging system comprises an optical coherence tomography system, and wherein the sampling energy comprises a broad-spectrum light including at least one of near infrared or infrared light. Similarly, in at least some implementations, the elongated portion of the medical instrument comprises at least one of a needle or a catheter, and wherein the medical procedure includes a venipuncture or a catheterization. In at least some particular implementations, the scanning axis is collinear with the longitudinal axis of the elongated portion, the deflection angle is sixty degrees, and wherein the stage assembly rotates the tip portion at 0.5 Hz and reciprocates the tip portion along a translation range of 6 mm. Similarly, in at least some particular implementations, displaying information including one or more relative positions of the target tissue relative to the elongated portion of the medical instrument comprises displaying visual image information including one or more of a roll position view, an alignment position view, and a pitch position view.

Furthermore, in at least some implementations, an apparatus for performing a medical procedure, comprising: a medical instrument including an elongated portion configured to be inserted into a body portion of a patient and having an optical fiber at least partially disposed within the elongated portion, the optical fiber having a tip portion that is extendable beyond a distal end of the elongated portion; an imaging system configured to provides a sampling energy into the optical fiber, the sampling energy being emitted from the tip portion along a sampling vector at a deflection angle with the longitudinal axis into one or more tissues of the body portion as the elongated portion is inserted into the body portion; and a stage assembly to actuate the tip portion to perform a scanning of the one or more tissues with the sampling energy emitted from the tip portion, the stage assembly being configured to rotate at least the tip portion about a scanning axis that is parallel with the longitudinal axis and to reciprocate at least the tip portion along the scanning axis that is parallel with the longitudinal axis with the tip portion proximate to and extended beyond the distal end of the elongated portion, the sampling energy being emitted from the tip portion in a scanning pattern. The imaging system may be further configured to receive a reflected energy that is reflected from the one or more tissues back through the optical fiber, the reflected energy providing a plurality of one-dimensional arrays of intensity values of the reflected energy at a plurality of distances along the sampling vector. The apparatus further comprises a control system configured to analyze the plurality of one-dimensional arrays of intensity values of the reflected energy to determine a shape of a target tissue and a location of the target tissue relative to the elongated portion of the medical instrument, and display information including one or more relative positions of the target tissue relative to the elongated portion of the medical instrument for guiding the medical instrument into engagement with the target tissue as the elongated portion is inserted into the body portion for performance of the medical procedure.

In at least some implementations, the imaging system is configured to continuously receive the reflected energy, and the control system is configured to continuously analyze the reflected energy and display the information for guiding the medical instrument into engagement with the target tissue in approximately real-time during advancement of the distal end of the elongated portion toward the target tissue during the medical procedure.

Similarly, in at least some implementations, the control system is configured to analyze the plurality of one-dimensional arrays of intensity values of the reflected energy, including determining an axis, a radius, and at least one point on the axis of a cylindrical shape of the target tissue. In still other implementations, the control system is configured to analyze the plurality of one-dimensional arrays of intensity values of the reflected energy, including determining a shape of the target tissue as a quadric surface and performing a least-squares fit of the intensity values onto the quadric surface. In further implementations, the control system is configured to determine that the distal end of the elongated portion had contacted the target tissue, and to control the stage assembly to retract the tip portion of the optical fiber into the distal end and at least temporarily suspend at least the reciprocating of the tip portion along the longitudinal axis until the distal end of the elongated portion has penetrated an outer wall of the target tissue. And in further implementations, the scanning axis is collinear with the longitudinal axis of the elongated portion, and the imaging system comprises an optical coherence tomography system, and wherein the sampling energy comprises a broad-spectrum light including at least one of near infrared or infrared light.

In addition, in at least some implementations, a system for performing a medical procedure comprises one or more processors, and one or more memory devices operatively coupled to the one or more processors and bearing one or more instructions that, when executed by the one or more processors, perform operations including: actuating an imaging system to provide a sampling energy when a medical instrument is engaged with a body portion of a patient, the medical instrument including an elongated portion configured to be inserted into the body portion and having an optical fiber at least partially disposed within the elongated portion, the optical fiber having a tip portion that is extendable beyond a distal end of the elongated portion, the sampling energy being provided into the optical fiber and emitted from the tip portion along a sampling vector at a deflection angle with the longitudinal axis into one or more tissues of the body portion as the elongated portion is inserted into the body portion; actuating a stage assembly to move the tip portion to perform a scanning of the one or more tissues with the sampling energy emitted from the tip portion, the stage assembly rotating at least the tip portion about a scanning axis that is parallel with the longitudinal axis and reciprocating at least the tip portion along the scanning axis that is parallel with the longitudinal axis with the tip portion proximate to and extended beyond the distal end of the elongated portion, the sampling energy being emitted from the tip portion in a scanning pattern; receiving a reflected energy that is reflected from the one or more tissues back through the optical fiber to the imaging system, the reflected energy providing a plurality of one-dimensional arrays of intensity values of the reflected energy at a plurality of distances along the sampling vector; analyzing the plurality of one-dimensional arrays of intensity values of the reflected energy to determine a shape of a target tissue and a location of the target tissue relative to the elongated portion of the medical instrument; and displaying information including one or more relative positions of the target tissue relative to the elongated portion of the medical instrument for guiding the medical instrument into engagement with the target tissue as the elongated portion is inserted into the body portion for performance of the medical procedure.

In at least some implementations, the operations comprise analyzing the plurality of one-dimensional arrays of intensity values of the reflected energy, including determining an axis, a radius, and at least one point on the axis of a cylindrical shape of the target tissue. And in still other implementations, the operations comprise analyzing the plurality of one-dimensional arrays of intensity values of the reflected energy, including determining a shape of the target tissue as a quadric surface and performing a least-squares fit of the intensity values onto the quadric surface.

In the foregoing description, many specific details of certain implementations are described and shown in the accompanying figures. One skilled in the art will understand that the present disclosure may have other possible implementations, and that such other implementations may be practiced with or without some of the particular details set forth in the foregoing description. In addition, it will be appreciated that although various aspects may be described in a particular order, or with respect to certain figures or certain embodiments, it should be appreciated that such aspects may be variously combined or re-ordered to create alternate implementations that remain consistent with the scope of the present disclosure and the claims set forth below.

It should be appreciated that the particular embodiments of processes described herein are merely particular implementations of the present disclosure, and that the present disclosure is not limited to the particular implementations described herein and shown in the accompanying figures. In addition, in alternate implementations, certain acts need not be performed in the order described, and may be modified or combined, and/or may be omitted entirely, depending on the circumstances. Moreover, in various implementations, the acts described may be implemented by a computer, controller, processor, programmable device, or any other suitable device, and may be based on instructions stored on one or more computer-readable media or otherwise stored or programmed into such devices. In the event that computer-readable media are used, the computer-readable media can be any available media that can be accessed by a device to implement the instructions stored thereon.

Various methods, systems, and techniques have been described herein in the general context of computer-executable instructions, such as program modules, executed by one or more processors or other devices. Generally, program modules include routines, programs, objects, components, data structures, etc. that perform particular tasks or implement particular abstract data types. Typically, the functionality of the program modules may be combined or distributed as desired in various alternate embodiments. In addition, embodiments of these methods, systems, and techniques may be stored on or transmitted across some form of computer readable media.

The foregoing examples are meant to be illustrative only, and omission of an example here should not be construed as intentional or intentionally disavowing subject matter. The scope of the invention set forth herein is defined solely by the following claims at the end of this application.

In a general sense, those skilled in the art will recognize that the various aspects described herein which can be implemented, individually and/or collectively, by a wide range of hardware, software, firmware, and/or any combination thereof. Those having skill in the art will recognize that the subject matter described herein may be implemented in an analog or digital fashion or some combination thereof.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

Throughout this application, examples and lists are given, and these examples and/or lists may be delineated with parentheses, commas, the abbreviation “e.g.,” or some combination thereof. Unless explicitly otherwise stated, these examples and lists are merely exemplary and are non-exhaustive. In most cases, it would be prohibitive to list every example and every combination. Thus, smaller, illustrative lists and examples are used, with focus on imparting understanding of the claim terms rather than limiting the scope of such terms.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

Although one or more users maybe shown and/or described herein, and other places, as a single illustrated figure, those skilled in the art will appreciate that one or more users may be representative of one or more human users, robotic users (e.g., computational entity), and/or substantially any combination thereof (e.g., a user may be assisted by one or more robotic agents) unless context dictates otherwise. Those skilled in the art will appreciate that, in general, the same may be said of “sender” and/or other entity-oriented terms as such terms are used herein unless context dictates otherwise.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

Throughout this application, the terms “in an embodiment,” “in at least some embodiments,” “in one embodiment,” “in some embodiments,” “in several embodiments,” “in at least one embodiment,” “in various embodiments,” and the like, may be used. Each of these terms, and all such similar terms should be construed as “in at least one embodiment, and possibly but not necessarily all embodiments,” unless explicitly stated otherwise. Specifically, unless explicitly stated otherwise, the intent of phrases like these is to provide non-exclusive and non-limiting examples of implementations of the invention. The mere statement that one, some, or may embodiments include one or more things or have one or more features, does not imply that all embodiments include one or more things or have one or more features, but also does not imply that such embodiments must exist. It is a mere indicator of an example and should not be interpreted otherwise, unless explicitly stated as such.

Throughout this application, the terms “in an implementation,” “in at least some implementations,” “in one implementation,” “in some implementations,” “in several implementations,” “in at least one implementation,” “in various implementations,” and the like, may be used. Each of these terms, and all such similar terms should be construed as “in at least one implementation, and possibly but not necessarily all implementations,” unless explicitly stated otherwise. Specifically, unless explicitly stated otherwise, the intent of phrases like these is to provide non-exclusive and non-limiting examples of implementations of the invention. The mere statement that one, some, or may implementations include one or more things or have one or more features, does not imply that all implementations include one or more things or have one or more features, but also does not imply that such implementations must exist. It is a mere indicator of an example and should not be interpreted otherwise, unless explicitly stated as such.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

What is claimed is:

1. A method for performing a medical procedure, comprising:
engaging a medical instrument with a body portion of a patient, the medical instrument including an elongated portion configured to be inserted into the body portion and having an optical fiber at least partially disposed within the elongated portion, the optical fiber having a tip portion that is extendable beyond a distal end of the elongated portion;
actuating an imaging system that provides a sampling energy into the optical fiber, the sampling energy being emitted from the tip portion along a sampling vector at a deflection angle with the longitudinal axis into one or more tissues of the body portion as the elongated portion is inserted into the body portion, the one or more tissues including a target tissue;
actuating a stage assembly to move the tip portion to perform a scanning of the one or more tissues with the sampling energy emitted from the tip portion, the stage assembly rotating at least the tip portion about a scanning axis that is parallel with the longitudinal axis and reciprocating at least the tip portion along the scanning axis that is parallel with the longitudinal axis with the tip portion proximate to and extended beyond the distal end of the elongated portion, the sampling energy being emitted from the tip portion in a scanning pattern;
receiving a reflected energy that is reflected from the one or more tissues back through the optical fiber to the imaging system, the reflected energy providing a plurality of one-dimensional arrays of intensity values of the reflected energy at a plurality of distances along the sampling vector;
analyzing the plurality of one-dimensional arrays of intensity values of the reflected energy to determine a shape of the target tissue and a location of the target tissue relative to the elongated portion of the medical instrument; and
displaying information including one or more relative positions of the target tissue relative to the elongated portion of the medical instrument for guiding the medical instrument into engagement with the target tissue as the elongated portion is inserted into the body portion for performance of the medical procedure.

2. The method of claim 1, further comprising advancing the distal end of the elongated portion toward the target tissue; and
wherein the receiving of the reflected energy, the analyzing of the reflected energy, and the displaying of the information for guiding the medical instrument into engagement with the target tissue are continuously performed in approximately real-time during the advancing of the distal end of the elongated portion toward the target tissue.

3. The method of claim 1, wherein analyzing the plurality of one-dimensional arrays of intensity values of the reflected energy to determine a shape of a target tissue and a location of the target tissue relative to the elongated portion of the medical instrument comprises approximating a shape of the target tissue as a cylinder, including determining an axis, a radius, and at least one point on the axis of the cylinder.

4. The method of claim 1, wherein analyzing the plurality of one-dimensional arrays of intensity values of the reflected energy to determine a shape of a target tissue and a location of the target tissue relative to the elongated portion of the medical instrument comprises determining a shape of the target tissue as a quadric surface and performing a least-squares fit of the intensity values onto the quadric surface.

5. The method of claim 1, wherein the scanning pattern includes a conical scanning pattern, and wherein analyzing the plurality of one-dimensional arrays of intensity values comprises analyzing the plurality of one-dimensional arrays of intensity values to determine an intersection of a cylindrical target tissue with the conical scanning pattern.

6. The method of claim 1, wherein the scanning pattern includes a helical scanning pattern, and wherein analyzing the plurality of one-dimensional arrays of intensity values comprises analyzing the plurality of one-dimensional arrays of intensity values to determine an intersection of a cylindrical target tissue with the helical scanning pattern.

7. The method of claim 1, further comprising:
when the distal end of the elongated portion contacts the target tissue, retracting the tip portion of the optical fiber into the distal end and at least temporarily suspending at least the reciprocating of the tip portion along the longitudinal axis until the distal end of the elongated portion penetrates an outer wall of the target tissue.

8. The method of claim 1, wherein the imaging system comprises an optical coherence tomography system, and wherein the sampling energy comprises a broad-spectrum light including at least one of near infrared or infrared light.

9. The method of claim 1, wherein the elongated portion of the medical instrument comprises at least one of a needle or a catheter, and wherein the medical procedure includes a venipuncture or a catheterization.

10. The method of claim 1, wherein the scanning axis is collinear with the longitudinal axis of the elongated portion, the deflection angle is sixty degrees, and wherein the stage assembly rotates the tip portion at 0.5 Hz and reciprocates the tip portion along a translation range of 6 mm.

11. The method of claim 1, wherein displaying information including one or more relative positions of the target tissue relative to the elongated portion of the medical instrument comprises displaying visual image information including one or more of a roll position view, an alignment position view, and a pitch position view.

12. An apparatus for performing a medical procedure, comprising:
a medical instrument including an elongated portion configured to be inserted into a body portion of a patient and having an optical fiber at least partially disposed within the elongated portion, the optical fiber having a tip portion that is extendable beyond a distal end of the elongated portion;
an imaging system configured to provides a sampling energy into the optical fiber, the sampling energy being emitted from the tip portion along a sampling vector at a deflection angle with the longitudinal axis into one or more tissues of the body portion as the elongated portion is inserted into the body portion;
a stage assembly to actuate the tip portion to perform a scanning of the one or more tissues with the sampling energy emitted from the tip portion, the stage assembly being configured to rotate at least the tip portion about a scanning axis that is parallel with the longitudinal axis and to reciprocate at least the tip portion along the scanning axis that is parallel with the longitudinal axis with the tip portion proximate to and extended beyond the distal end of the elongated portion, the sampling energy being emitted from the tip portion in a scanning pattern;
the imaging system being further configured to receive a reflected energy that is reflected from the one or more tissues back through the optical fiber, the reflected energy providing a plurality of one-dimensional arrays of intensity values of the reflected energy at a plurality of distances along the sampling vector;
a control system configured to
analyze the plurality of one-dimensional arrays of intensity values of the reflected energy to determine a shape of a target tissue and a location of the target tissue relative to the elongated portion of the medical instrument, and
display information including one or more relative positions of the target tissue relative to the elongated portion of the medical instrument for guiding the medical instrument into engagement with the target tissue as the elongated portion is inserted into the body portion for performance of the medical procedure.

13. The apparatus of claim 12, wherein the imaging system is configured to continuously receive the reflected energy, and the control system is configured to continuously analyze the reflected energy and display the information for guiding the medical instrument into engagement with the target tissue in approximately real-time during advancement of the distal end of the elongated portion toward the target tissue during the medical procedure.

14. The apparatus of claim 12, wherein the control system is configured to analyze the plurality of one-dimensional arrays of intensity values of the reflected energy, including determining an axis, a radius, and at least one point on the axis of a cylindrical shape of the target tissue.

15. The apparatus of claim 12, wherein the control system is configured to analyze the plurality of one-dimensional arrays of intensity values of the reflected energy, including determining a shape of the target tissue as a quadric surface and performing a least-squares fit of the intensity values onto the quadric surface.

16. The apparatus of claim 12, wherein the control system is configured to determine that the distal end of the elongated portion had contacted the target tissue, and to control the stage assembly to retract the tip portion of the optical fiber into the distal end and at least temporarily suspend at least the reciprocating of the tip portion along the longitudinal axis until the distal end of the elongated portion has penetrated an outer wall of the target tissue.

17. The apparatus of claim 12, wherein the scanning axis is collinear with the longitudinal axis of the elongated portion, and wherein the imaging system comprises an optical coherence tomography system, and wherein the sampling energy comprises a broad-spectrum light including at least one of near infrared or infrared light.

18. A system for performing a medical procedure, comprising:
one or more processors: and
one or more memory devices operatively coupled to the one or more processors and bearing one or more instructions that, when executed by the one or more processors, perform operations including:
actuating an imaging system to provide a sampling energy when a medical instrument is engaged with a body portion of a patient, the medical instrument including an elongated portion configured to be inserted into the body portion and having an optical fiber at least partially disposed within the elongated portion, the optical fiber having a tip portion that is extendable beyond a distal end of the elongated portion, the sampling energy being provided into the optical fiber and emitted from the tip portion along a sampling vector at a deflection angle with the longitudinal axis into one or more tissues of the body portion as the elongated portion is inserted into the body portion;
actuating a stage assembly to move the tip portion to perform a scanning of the one or more tissues with the sampling energy emitted from the tip portion, the stage assembly rotating at least the tip portion about a scanning axis that is parallel with the longitudinal axis and reciprocating at least the tip portion along the scanning axis that is parallel with the longitudinal axis with the tip portion proximate to and extended beyond the distal end of the elongated portion, the sampling energy being emitted from the tip portion in a scanning pattern;
receiving a reflected energy that is reflected from the one or more tissues back through the optical fiber to the imaging system, the reflected energy providing a plurality of one-dimensional arrays of intensity values of the reflected energy at a plurality of distances along the sampling vector;
analyzing the plurality of one-dimensional arrays of intensity values of the reflected energy to determine a shape of a target tissue and a location of the target tissue relative to the elongated portion of the medical instrument; and
displaying information including one or more relative positions of the target tissue relative to the elongated portion of the medical instrument for guiding the medical instrument into engagement with the target tissue as the elongated portion is inserted into the body portion for performance of the medical procedure.

19. The system of claim 18, wherein the operations comprise analyzing the plurality of one-dimensional arrays of intensity values of the reflected energy, including determining an axis, a radius, and at least one point on the axis of a cylindrical shape of the target tissue.

20. The system of claim 18, wherein the operations comprise analyzing the plurality of one-dimensional arrays of intensity values of the reflected energy, including determining a shape of the target tissue as a quadric surface and performing a least-squares fit of the intensity values onto the quadric surface.
